(19) United States
(12) Patent Application Publication
(10) Pub. No.: US 2006/0167110 A1
(43) Pub. Date: Jul. 27, 2006

(54) Methods for treating cerebrovascular disease by administering desmethylselegiline

(76) Inventors: Cherly D. Blume, Tampa, FL (US); Anthony R. DiSanto, Gobles, MI (US)

Correspondence Address: Margaret J. Sampson;VINSON & ELKINS LLP, First City Tower, 1001 Fannin Street, Suite 2300, Houston, TX, 77002-6760, US

(21) Appl. No.: 11/290,772

(22) Filed: Nov. 30, 2005

Related U.S. Application Data
(63) Continuation-in-part of application No. 10/885,221, filed on Jul. 6, 2004.
Application No. 10/885,221 is a continuation of application No. 10/251,727, filed on Sep. 20, 2002, now Pat. No. 6,759,053.
Application No. 10/251,727 is a continuation of application No. 09/800,022, filed on Mar. 5, 2001, now Pat. No. 6,455,060.
Application No. 09/800,022 is a division of application No. 09/448,483, filed on Nov. 24, 1999, now Pat. No. 6,210,706.
Application No. 09/448,483 is a division of application No. 08/679,328, filed on Jul. 12, 1996, now Pat. No. 6,033,682.
(63) Continuation-in-part of application No. 10/790,658, filed on Mar. 1, 2004.
Application No. 10/790,658 is a continuation of application No. 10/026,159, filed on Dec. 21, 2001, now Pat. No. 6,699,495.
Application No. 10/026,159 is a continuation of application No. 08/679,330, filed on Jul. 12, 1996, now Pat. No. 6,348,208.
Application No. 08/679,330 is a continuation-in-part of application No. PCT/US96/01561, filed on Jan. 11, 1996.
Application No. 08/679,330 is a continuation-in-part of application No. 08/372,139, filed on Jan. 13, 1995, now abandoned.
(60) Provisional application No. 60/001,979, filed on Jul. 31, 1995.

(51) Int. Cl. A61K 31/137; A61K 31/727; A61K 31/60

(52) U.S. Cl. 514/649; 514/56; 514/165

(57) ABSTRACT

The present disclosure is directed to methods for reducing the neuronal damage associated with cerebrovascular disease, such as stroke or cerebral edema, by administering R(−)-desmethylselegiline, S(+) desmethylselegiline, or a combination of the two. The cerebrovascular disease may be caused by ischemia or hypoxia.

### CROSS REFERENCES TO RELATED APPLICATIONS

The present application is a continuation-in-part of U.S. Ser. No. 10/885,221, filed Jul. 6,2004, which is a continuation of U.S. Ser. No. 10/251, 727, filed Sep. 20, 2002, now U.S. Pat. No. 6,759,053, which is a continuation of U.S. Ser. No. 09/800,022, filed Mar. 5, 2001, now U.S. Pat. No. 6,455,060, which is a division of U.S. Ser. No. 09/448,483, filed Nov. 24, 1999, now U.S. Pat. No. 6,210,706, which is a division of U.S. Ser. No. 08/679,328, filed Jul. 12, 1996, now U.S. Pat. No. 6,033,682; and U.S. Ser. No. 10/790,658, filed Mar. 1, 2004, which is a continuation of U.S. Ser. No. 10/026,159, filed Dec. 21, 2001, now U.S. Pat. No. 6,699,495, which is a continuation of U.S. Ser. No. 08/679,330, filed Jul. 12, 1996, now U.S. Pat. No. 6,348,208; which are continuations-in-part of PCT/US96/01561, filed Jan. 11, 1996, Provisional No. 60/001,979, filed Jul. 31, 1995, and U.S. Ser. No. 08/372,139, filed Jan. 13, 1995; each of which is hereby incorporated by reference in its entirety for all purposes.

### STATEMENT REGARDING FEDERALLY SPONSORED RESEARCH OR DEVELOPMENT

Not applicable.

### REFERENCE TO A “Microfiche Appendix”

Not applicable. BACKGROUND OF THE INVENTION

1. Field of the Invention

The present invention relates to methods and pharmaceutical compositions for using the selegiline metabolite R(−)-desmethylselegiline (also referred to simply as “desmethylselegiline” or “R(−)DMS”) alone; its enantiomer ent-desmethylselegiline (also referred to as “S(+) desmethylselegiline” or “S(+)DMS”) alone; or a combination, such as, for example, a racemic mixture, of the two enantiomers. In particular, the present disclosure provides compositions and methods for using these agents to treat cerebrovascular disease, particularly for alleviating the symptoms and damage associated with stroke, ischemia, and hypoxia.

2. Description of Related Art

The generic term stroke refers to the abrupt impairment of brain function caused by a variety of pathologic changes involving one (focal) or several (multifocal) intracranial or extracranial blood vessels. Approximately 80% of strokes are caused by blood vessel occlusion with too little blood flow (ischemic stroke) to the affected area, and the remaining 20% consist of intracranial hemorrhages, which are nearly equally divided between hemorrhage into brain tissue (parenchymatous hemorrhage) and hemorrhage into the surrounding subarachnoid space (subarachnoid hemorrhage). Some strokes are caused by abnormalities in cerebral circulation. Cerebrovascular diseases can be classified according to whether they affect the brain's vascular supply either focally or diffusely. A general discussion of cerebrovascular diseases is found in Goldman: Cecil Textbook of Medicine, 21st ed., Chapters 469-471, W. B. Saunders Company, 2000. Currently, stroke is the third leading cause of medically related deaths and the second most frequent cause of neurologic morbidity in developed countries.

Cerebrovascular diseases may result in, for example, stroke, intracranial hemorrhage, cerebral hypoxia, cerebral ischemia, hemorrhagic lesion, subderal hematoma, aneurysm, physical injury or accident. The identification of new methods for treating cerebrovascular diseases has obvious value in alleviating the suffering, disability, and/or death of patients. Presently there are few therapies that are generally accepted for treatment of cerebrovascular diseases and for their consequences. Indeed, the only general treatments available for acute ischemic stroke are the administration of tissue plasminogen activator (tPA) within three hours of symptom onset or aspirin within 24 hours of symptom onset. Thus, the opportunity to treat a subject is greatly hindered by time constraints. In this regard, the process of brain damage occurs within several hours of focal vascular occlusion, and this damage is often irreversible. Therefore, any treatment that is able to slow down the nervous system damage caused by cerebrovascular disorders will provide practitioners with a greater window of time for treating subjects with other effective therapies, as well as improve the overall prospects of recovery for the subject.

Two distinct monoamine oxidase enzymes are known in the art: monoamine oxidase A (MAO-A) and monoamine oxidase B (MAO-B). The cDNAs encoding these enzymes show different promoter regions and distinct exon portions, indicating they are encoded independently at different gene positions and exist as unique proteins. In addition, analysis of the two proteins has shown differences in their respective amino acid sequences.

The first compound found to selectively inhibit MAO-B was (R)-N-α-dimethyl-N-2- propynylbenzeethanamine, also known as L-(−)-N-α-N-2-propynylphenethylamine, (−)-deprenil, L-(−)-deprenyl, R-(−)-deprenyl, or selegiline. Selegiline has the following structural formula:

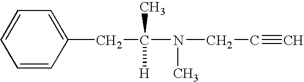

The various diseases and conditions for which selegiline is disclosed as being useful include: depression (U.S. Pat. No. 4,861,800); Alzheimer's disease and Parkinson's disease, particularly through the use of transdermal dosage forms, including ointments, creams and patches; macular degeneration (U.S. Pat. No. 5,242,950); age-dependent degeneracies, including renal function and cognitive function as evidenced by spatial learning ability (U.S. Pat. No. 5,151,449); pituitary-dependent Cushing's disease in humans and nonhumans (U.S. Pat. No. 5,192,808); immune system dysfunction in both humans (U.S. Pat. No. 5,387,615) and animals (U.S. Pat. No. 5,276,057); age-dependent weight loss in mammals (U.S. Pat. No. 5,225,446); and schizophrenia (U.S. Pat. No. 5,151,419). PCT Published Application WO 92/17169 discloses the use of selegiline in the treatment of neuromuscular and neurodegenerative disease and in the treatment of CNS injury due to hypoxia, hypoglycemia, ischemic stroke or trauma. In addition, the biochemical effects of selegiline on neuronal cells have been extensively studied. For example, see Tatton, et al., “Selegiline Can Mediate Neuronal Rescue Rather than Neuronal Protection,” Movement Disorders 8 (Supp. 1):S20-S30 (1993); Tatton, et al., “Rescue of Dying Neurons,” J. Neurosci. Res. 30:666-672 (1991); and Tatton, et al., “(−)Deprenyl Prevents Mitochondrial Depolarization and Reduces Cell Death in Trophically-Deprived Cells,” 11th Int'l Symp. on Parkinson's Disease, Rome, Italy, Mar. 26-30, 1994.

Knollema et al. has also reported that selegiline can be used as a prophylactic treatment for brain tissue in a rat model when it is administered before hypoxia/ischemia (stroke) (Knollema et al., Stroke, 26(10): 1883-87, 1995). Specifically, selegiline was found to exert protective effects against ischemic/hypoxic damage in the striatum and thalamus areas of the brain in rats. Although the mechanisms by which selegiline was able to give this observed protection were unclear, the protection may be due to MAO-B independent effects of selegiline.

Although selegiline is reported as being effective in treating the foregoing conditions, neither the precise number or nature of its mechanism or mechanisms of action are known. However, there is evidence that selegiline provides neuroprotection or neuronal rescue, possibly by reducing oxidative neuronal damage, increasing the amount of the enzyme superoxide dismutase, and/or reducing dopamine catabolism. For example, PCT Published Application WO 92/17169 reports that selegiline acts by directly maintaining, preventing loss of, and/or assisting in, the nerve function of animals.

Selegiline is known to be useful when administered to a subject through a wide variety of routes of administration and dosage forms. For example U.S. Pat. No. 4,812,481 (Degussa AG) discloses the use of concomitant selegiline-amantadine in oral, peroral, enteral, pulmonary, rectal, nasal, vaginal, lingual, intravenous, intraarterial, intracardial, intramuscular, intraperitoneal, intracutaneous, and subcutaneous formulations. U.S. Pat. No. 5,192,550 (Alza Corporation) describes a dosage form comprising an outer wall impermeable to selegiline but permeable to external fluids. This dosage form may have applicability for the oral, sublingual or buccal administration of selegiline. Similarly, U.S. Pat. No. 5,387,615 discloses a variety of selegiline compositions, including tablets, pills, capsules, powders, aerosols, suppositories, skin patches, parenterals, and oral liquids, including oil-aqueous suspensions, solutions, and emulsions. Also disclosed are selegiline-containing sustained release (long acting) formulations and devices.

Although a highly potent and selective MAO-B inhibitor, the use of selegiline can be limited by its dose-dependent specificity for MAO-B. The selectivity of selegiline in the inhibition of MAO-B is important to its safety profile following oral administration. Inhibition of MAO-A in peripheral sites (such as, for example, gastric epithelium, liver parenchyma, and sympathetic neurons) may cause toxic side effects by interfering with the metabolism of, for example, dietary tyramine. Tyramine is normally metabolized in the gastrointestinal tract by MAO-A, but when MAO-A is inhibited, tyramine absorption is increased following consumption of tyramine-containing foods such as cheese, beer, herring, etc. This results in the release of catecholamines which can precipitate a hypertensive reaction, referred to as the “cheese effect.” This effect is characterized by Goodman and Gilman as the most serious toxic effect associated with MAO-A inhibitors.

Selegiline is metabolized into its N-desmethyl analog and other metabolites. Structurally, this N-desmethyl metabolite is the R(−) enantiomeric form R(−)DMS of a secondary amine of the formula:

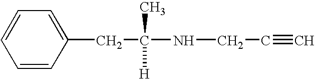

Heretofore, R(−)DMS was not known to have pharmaceutically useful MAO-related effects, i.e., potent and selective inhibitory effects on MAO-B. In the course of determining the usefulness of R(−)DMS for the purposes of the present invention, the MAO-related effects of R(−)DMS were more completely characterized. This characterization has established that desmethylselegiline has exceedingly weak MAO-B inhibitory effects and no advantages in selectivity with respect to MAO-B compared to selegiline.

For example, the present characterization established that selegiline has an IC₅₀ value against MAO-B in human platelets of 5×10⁻⁹ M whereas R(−)DMS has an IC₅₀ value of 4×10⁻⁷ M, indicating the latter is approximately 80 times less potent as an MAO-B inhibitor than the former. Similar characteristics can be seen in the following data measuring inhibition of MAO-B and MAO-A in rat cortex mitochondrial-rich fractions:

TABLE 1
Inhibition of MAO by Selegiline and Desmethylselegiline

| Percent Inhibition | |
|---|---|
| Selegiline | R(−)desmethylselegiline |

| Conc. | MAO-B | MAO-A | MAO-B | MAO-A |
|---|---|---|---|---|
| 0.003 μM | 16.70 | — | 3.40 | — |
| 0.010 μM | 40.20 | — | 7.50 | — |
| 0.030 μM | 64.70 | 0 | 4.60 | — |
| 0.100 μM | 91.80 | — | 6.70 | — |
| 0.300 μM | 94.55 | 9.75 | 26.15 | 0.0 |
| 1.000 μM | 95.65 | 32.55 | 54.73 | 0.70 |
| 3.000 μM | 98.10 | 65.50 | 86.27 | 4.10 |
| 10.000 μM | — | 97.75 | 95.15 | 11.75 |
| 30.000 μM | — | — | 97.05 | — |
| 100.000 μM | — | — | — | 56.10 |

As is apparent from the above table, selegiline is approximately 128 times more potent as an inhibitor of MAO-B relative to MAO-A, whereas R(−)DMS is about 97 times more potent as an inhibitor of MAO-B relative to MAO-A. Accordingly, R(−)DMS appears to have an approximately equal selectivity for MAO-B compared to MAO-A as-selegiline, albeit with a substantially reduced potency.

Analogous results are obtained in rat brain tissue. Selegiline exhibits an IC₅₀, for MAO-B of 0.11×10-7 M whereas R(−)DMS has an IC₅₀ value of 7.3×10⁻⁷ M, indicating R(−)DMS is approximately 70 times less potent as an MAO-B inhibitor than selegiline. Both compounds exhibit low potency in inhibiting MAO-A in rat brain tissue, 0.18×10⁻⁵ for selegiline, 7.0×10⁻⁵ for R(−)DMS. Thus, in vitro R(−)DMS is approximately 39 times less potent than selegiline in inhibiting MAO-A.

Based on its pharmacological profile as set forth above, R(−)DMS as an MAO-B inhibitor provides no advantages in either potency or selectivity compared to selegiline. Indeed, the above in vitro data suggest that use of R(−)DMS as an MAO-B inhibitor requires on the order of 70 times the amount of selegiline.

The potency of R(−)DMS as an MAO-B inhibitor in vivo has been reported by Heinonen, E. H., et al. (“[R(−)Desmethylselegiline, a metabolite of selegiline, is an irreversible inhibitor of MAO-B in human subjects,” referenced in Academic Dissertation “Selegiline in the Treatment of Parkinson's Disease,” from Research Reports from the Department of Neurology, University of Turku, Turku, Finland, No. 33 (1995), pp. 59-61). According to Heinonen, R(−)DMS in vivo has only about one-fifth the MAO-B inhibitory effect of selegiline, i.e., a dose of 10 mg of desmethylselegiline would be required for the same MAO-B effect as 1.8 mg of selegiline. In rats, Borbe reported R(−)DMS to be an irreversible inhibitor of MAO-B, with a potency about 60 fold lower than selegiline in vitro and about 3 fold lower ex vivo (Barbe, H. O., J Neural Trans. (Suppl.):32:131 (1990)). Thus, all these previous investigators have reported data indicating that R(−)DMS is a less-preferred, less effective MAO inhibitor than selegiline and therefore a less desirable therapeutic compound.

### BRIEF SUMMARY OF THE INVENTION

The present invention is based upon the surprising discovery that R(−)DMS and its enantiomer S(+)DMS, having the following structure: 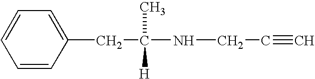
are particularly useful in providing selegiline-like effects in subjects, notwithstanding dramatically reduced MAO-B inhibitory activity and an apparent lack of enhanced selectivity for MAO-B compared to selegiline. Surprisingly, R(−)DMS, S(+)DMS, and combinations such as racemic mixtures of the two are able to reduce, alleviate, or eliminate in whole or in part the neuronal damage associated with cerebrovascular disease, such as stroke, cerebral ischemia, and cerebral hypoxia. In particular, the disclosure provides a method of protecting a patient from or treating a patient for cerebrovascular disease that results from damage to the brain caused by, for example, ischemia, stroke, transient ischemic attack, intracranial hemorrhage, occlusive hemorrhage, cerebral hemorrhage, subarachnoid hemorrhage, hypoxia, hemorrhagic lesion, subderal hematoma, aneurysm, mycotic aneurysm, venous occlusion, diffuse ischemia, cerebral abscess, physical injury, or accident, by administering R(−)DMS, S(+)DMS, or a combination of the two in an amount sufficient to treat, reduce, or eliminate one or more of the symptoms and damage associated with the cerebrovascular disease.

The administration of R(−)DMS, S(+)DMS, or a mixture of the two can also be used to slow the progressive damage caused by cerebrovascular diseases, which can provide practitioners a greater window of time for treating subjects with other effective therapies, such as aspirin, tPA, heparin, heparinoids, ticlopidine, clopidogrel, warfarin, glutamate receptor antagonists, sodium, potassium, channel blockers, antioxidants, anti-inflammatory compounds, nimodipine, phenylephrine, dopamine, or growth factors. Typically, the subject or patient will be a human.

The present disclosure provides novel pharmaceutical compositions in which R(−)DMS, S(+)DMS, or a combination, such as a racemic mixture, of the two is employed as the active ingredient. Also provided are novel therapeutic methods involving the administration of such compositions. More specifically, the present invention provides:

(1) A pharmaceutical composition comprising an amount of R(−)DMS, S(+)DMS, or a combination of the two, such that one or more unit doses of the composition administered on a periodic basis is effective to treat, in whole or in part, the damage associated with cerebrovascular disease in a subject to whom the unit dose or unit doses are administered. This composition may be formulated for non-oral or oral administration.

(2) A method of treating the damage associated with cerebrovascular disease in a subject, such as a mammal, which comprises administering to the subject R(−)DMS, S(+)DMS, or a combination of the two, in a dosage regimen effective to treat, ameliorate, reduce, or eliminate, in whole or in part, the symptoms, progression, and/or neuronal damage associated with cerebrovascular disease, such as a daily dose, administered in a single or multiple dosage regimen of at least about 0.0015 mg, calculated on the basis of the free secondary amine, per kg of the mammal's body weight.

(3) A transdermal delivery system for use in treating the damage associated with cerebrovascular disease in a subject which comprises a layered composite of one or more layers with at least one layer including an amount of R(−)DMS, S(+)DMS, or a combination of the two sufficient to supply a daily transdermal dose of at least about 0.0015 mg of the free secondary amine, per kg of the mammal's body weight.

(4) A therapeutic package for dispensing to, or for use in dispensing to, a subject being treated for the damage associated with cerebrovascular disease. The package contains one or more unit doses, each such unit dose comprising an amount of R(−)DMS, S(+)DMS, or a combination of the two, such that periodic administration is effective in treating the subject's disorder. The therapeutic package also comprises a finished pharmaceutical container containing the unit doses of R(−)DMS, S(+)DMS, or combination thereof, and further containing or comprising labeling directing the use of the package in the treatment of damage associated with cerebrovascular disease. The unit doses may be adapted for oral administration, e.g. as tablets or capsules, or may be adapted for non-oral administration.

(5) A method of dispensing R(−)DMS, S(+)DMS, or a combination of the two, to a patient being treated for the damage associated with cerebrovascular disease. The method comprises providing patients with a therapeutic package having one or more unit doses of desmethylselegiline, ent-desmethylselegiline, or a mixture of the two, in an amount such that administration to the patient is effective in treating cerebrovascular disease. The package also comprises a finished pharmaceutical container containing the desmethylselegiline, ent-desmethylselegiline, or a mixture of the two, and having labeling directing the use of the package in the treatment of damage associated with cerebrovascular disease. The unit doses in the package may be adapted for either oral or non-oral use.

Preferred embodiments of the present disclosure are methods for preventing or treating damage to the brain caused by, for example, ischemia, stroke, transient ischemic attack, intracranial hemorrhage, occlusive hemorrhage, cerebral hemorrhage, subarachnoid hemorrhage, hypoxia, hemorrhagic lesion, subderal hematoma, aneurysm, mycotic aneurysm, venous occlusion, diffuse ischemia, cerebral abscess, physical injury, or accident; in a subject in need of such prevention or treatment, by administering to the subject R(−)-desmethylselegiline, S(+)-desmethylselegiline, or a mixture of R(−)-desmethylselegiline and S(+)-desmethylselegiline. Preferably the desmethylselegiline enantiomer or enantiomers are administered in an amount sufficient to prevent, reduce, or eliminate one or more of the symptoms associated with the condition. In a preferred embodiment, the subject is a mammal, more preferably a human or a domesticated animal.

Another preferred embodiment of the present disclosure is a method for treating a subject's tissue damage associated with cerebrovascular disease comprising:
a) administering to the subject an agent known to have a therapeutic effect on a cerebrovascular disease, wherein the agent is administered at a dose effective at reducing or eliminating the progression of the cerebrovascular event;
b) concurrently administering R(−)-desmethylselegiline, S(+)-desmethylselegiline, or a mixture of R(−)-desmethylselegiline and S(+)-desmethylselegiline to the patient at a dose effective at reducing or eliminating the progression of the damage associated with cerebrovascular disease.

In a preferred embodiment, the large-fiber peripheral neuropathy is a large-fiber sensory neuropathy or a large-fiber motor neuropathy, that results from abnormal function or pathological change in large, myelinated axons. In another preferred embodiment, the small-fiber peripheral neuropathy results from abnormal function or pathological change in small, myelinated axons, or small, unmyelinated axons. In yet another preferred embodiment, the autonomic peripheral neuropathy results from the dysfunction of peripheral autonomic nerves, and preferably the peripheral autonomic nerves involved are small, myelinated nerves.

In preferred embodiments, R(−)-desmethylselegiline or S(+)-desmethylselegiline is administered in a substantially enantiomerically pure form. In other preferred embodiments, R(−)-desmethylselegiline and/or S(+)-desmethylselegiline are administered as the free base or as an acid addition salt. Preferably the acid addition salt is hydrochloride salt. In yet another preferred embodiment, the R(−)-desmethylselegiline, S(+)-desmethylselegiline, or combination of the two is administered orally or non-orally. Preferably, the desmethylselegiline enantiomers are administered by a route that avoids absorption of the desmethylselegiline enantiomers from the gastrointestinal tract. Preferable routs of non-oral administration are transdermal, buccal, sublingual, parenteral and intravenous. In yet another preferred embodiment, R(−)-desmethylselegiline and/or S(+)-desmethylselegiline are administered at a dose of between 0.01 mg/kg per day and 0.15 mg/kg per day based upon the weight of the free amine.

Another preferred embodiment of the present disclosure is a pharmaceutical composition that includes R(−)-desmethylselegiline, S(+)-desmethylselegiline, or a mixture of R(−)-desmethylselegiline and S(+)-desmethylselegiline, as well as a second therapeutic agent useful in the treatment of cerebrovascular disease or its consequences. In a preferred embodiment, one or more therapeutic agents are included in the pharmaceutical composition. In another preferred embodiment, the R(−)-desmethylselegiline, S(+)-desmethylselegiline, or combination of R(−)-desmethylselegiline and S(+)-desmethylselegiline, and the second therapeutic agent, are present in the pharmaceutical composition in an amount such that one or more unit doses of the composition are effective to treat, prevent, reduce, or eliminate the damage associated with cerebrovascular disease in a subject. In other preferred embodiments, R(−)DMS and/or S(+)DMS are administered as the free base or as an acid addition salt. Preferably the acid addition salt is hydrochloride salt. In another preferred embodiment of the present disclosure, the second therapeutic agent useful in the treatment of damage associated with cerebrovascular disease is selected from the group consisting of aspirin, tPA; heparin; low-molecular-weight heparins; heparinoids; ticlopidine; clopidogrel;, warfarin; glutamate receptor antagonists; sodium, potassium, and channel blockers; antioxidants; anti-inflammatory compounds; nimodipine; phenylephrine; dopamine; and growth factors.

In other preferred embodiments, the R(−)DMS, S(+)DMS, or combination of the two enantioners in a unit dose of the pharmaceutical composition is between about 0.015 and about 5.0 mg/kg, more preferably between about 0.6 and about 0.8 mg/kg, calculated on the basis of the free secondary amine. In another preferred embodiment, the R(−)DMS, S(+)DMS, or combination of the two enantioners in a unit dose of the pharmaceutical composition is between about 1.0 mg and about 100.0 mg, more preferably between about 5.0 mg and about 10.0 mg. In yet another preferred embodiment, the pharmaceutical composition is for oral administration, for non-oral administration, or for transdermal administration. In a preferred embodiment the pharmaceutical composition is a transdermal patch.

### BRIEF DESCRIPTION OF THE SEVERAL VIEWS OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: HPLC Chromatogram of Purified R(−)DMS (Microsorb MV Cyano Column). The purity of a preparation of R(−)DMS was determined by HPLC on a Microsorb MV Cyano column and results are shown in FIG. 1. The column had dimensions of 4.6 mm×15 cm. and was developed at a flow rate of 1.0 ml/min using a mobile phase containing 90% 0.01 M H₃PO₄ (pH 3.5) and 10% acetonitrile. The column was run at a temperature of 40° C. and effluent was monitored at a wavelength of 215 nm. The chromatogram shows one major peak appearing at a time of 6.08 minutes and having 99.5% of the total light-absorbing material eluted from the column. No other peak had greater than 0.24%.

FIG. 2: HPLC Elution Profile of R(−)DMS (Zorbax Mac-Mod C 18 Column). The same preparation that was analyzed in the experiments discussed in FIG. 1 was also analyzed for purity by HPLC on a Zorbax Mac-Mod SB-C 18 column (4.6 mm×75 mm). Effluent was monitored at 215 nm and results can be seen in FIG. 2. Greater than 99.6% of the light-absorbing material appeared in the single large peak eluting at a time of between 2 and 3 minutes.

FIG. 3: Mass Spectrum of R(−)DMS. A mass spectrum was obtained for purified R(−)DMS and results are shown in FIG. 3. The spectrum is consistent with a molecule having a molecular weight of 209.72 amu and a molecular formula of C₁₂H₁₅N—HCl.

FIG. 4: Infrared Spectrum. (KBr) of Purified R(−)DMS. Infrared spectroscopy was performed on a preparation of R(−)DMS and results are shown in FIG. 4. The solvent used was CDCl₃.

FIG. 5: NMR Spectrum of Purified R(−)DMS. A preparation of purified R(−)DMS was dissolved in CDCl₃ and ¹H NMR spectroscopy was performed at 300 nm. Results are shown in FIG. 5.

FIG. 6: HPLC Chromatogram of S(+)DMS. The purity of a preparation of S(+)DMS was examined by reverse phase HPLC on a 4.6 min×75 min Zorbax Mac-Mod SB-C18 column. The elution profile, monitored at 215 nm, is shown in FIG. 6. One major peak appears in the profile at a time of about 3 minutes and contains greater than 99% of the total light-absorbing material that eluted from the column.

FIG. 7: Mass Spectrum of Purified S(+)DMS. Mass spectroscopy was performed on the same preparation examined in FIG. 6. The spectrum is shown in FIG. 7 and is consistent with the structure of S(+)DMS.

FIG. 8: Infrared Spectrum (KBr) of Purified S(+)DMS. The preparation of S(+)DMS discussed in connection with FIGS. 6 and 7 was examined by infrared spectroscopy and results are shown in FIG. 8.

FIG. 9: Effect of Selegiline on Neuron Survival. Mesencephalic cultures were prepared from embryonic 14 day rats. Cultures were used at about 1.5 million cells per plate and were maintained either in growth medium alone (control cultures) or in growth medium supplemented with selegiline. Complete medium changes were performed daily to induce excitotoxic damage to the neurons. Twenty-four hours following the last medium change, cells were immunostained for the presence of tyrosine hydroxylase (“TH”), a marker of neurons containing catecholamine as the neurotransmitter. Striped bars represent results obtained for cultures maintained in the presence of selegiline and open bars represent results for control cultures. In all cases, results are expressed as a percentage of TH positive cells present in control cultures assayed on day 1. The abbreviation “DIV” refers to “days in vitro.” Asterisks or stars above bars both in FIG. 9 and the figures discussed below indicate a result that differs from controls in an amount that is statistically significant, i.e. p <0.05.

FIG. 10: [³H]-Dopamine Uptake by surviving Mesencephalic Cells. Cells, cultured as described above for FIG. 9, were tested for their uptake of labeled dopamine (a catecholamine) and results are shown in FIG. 10. Striped bars represent uptake in cells maintained in the presence of selegiline and open bars represent uptake in control cultures.

FIG. 11: Effect of Selegiline on Glutamate Receptor Dependent Neuronal Cell Death. Rat embryonic mesencephalic cells were cultured as described above. After allowing cultures to stabilize, the culture medium was changed daily for a period of 4 days to induce glutamate receptor-dependent cell death. Depending on the culture, medium contained either 0.5, 5.0 or 50 μM selegiline. After the final medium change, cultured cells were immunostained for the presence of tyrosine hydroxylase. From left to right, bars represent results for controls, 0.5, 5.0 and 50 μM selegiline.

FIG. 12: Effect of Selegiline on Dopamine Uptake in Neuronal Cultures. Rat mesencephalic cells were cultured and medium was changed on a daily basis as discussed for FIG. 11. Uptake of tritiated dopamine by cells was measured and results are shown in the figure. From left to right, bars are in the same order as for FIG. 11.

FIG. 13: Effect of R(−)Desmethylselegiline on Glutamate Receptor Dependent Neuronal Cell Death. Rat embryonic mesencephalic cultures were prepared as described above except that R(−)DMS was used instead of selegiline. On day 9, the number of TH positive cells remaining in cultures was determined. Results are expressed as a percentage of control. From left to right, bars show results for controls, 0.5, 5 and 50 μM R(−)DMS.

FIG. 14: Effect of R(−)Desmethylselegiline on Dopamine Uptake by Surviving Neurons in Cultures. Cell cultures were prepared as described above for FIG. 13 and then tested for uptake of tritiated dopamine. Results for controls and for cells maintained in the presence of 0.5 μM, 5 μM and 50 μM desmethylselegiline are shown from left to right in the figure.

FIG. 15: Comparison of Dopamine Uptake in Surviving Mesencephalic Cells Incubated in the Presence of Different Monoamine Oxidase Inhibitors. Rat embryonic mesencephalic cells were prepared as described for FIGS. 11-14 and incubated in the presence of a variety of monoamine oxidase inhibitors. The inhibitors examined were selegiline; R(−) desmethylselegiline; pargyline; and clorgyline, all at concentrations of 0.5, 5 and 50 μM. In addition, cells were incubated in the presence of the glutamate receptor blocker MK-801 at a concentration of 10 μM. Cultures were tested for uptake of tritiated dopamine as a marker of cell survival.

FIG. 16: Relative Effectiveness of R(−)DMS and S(+)DMS in Maintaining [³H]-Dopamine Uptake by Surviving Cultured Mesencephalic Cells (NMDA Model). Concentrations of R(−)DMS and S(+)DMS were assayed for their effect on [³H]-dopamine uptake by cultured rat mesencephalic cells exposed to the toxin N-methyl-D-aspartate (NMDA). Results were expressed as a percentage of the uptake seen in control cultures not exposed to NMDA and are shown in FIG. 16. From the left, the bars represent: cells incubated with medium alone; medium+5 μM deprenyl; medium+0.5 μM R(−)DMS; medium+5 μM R(−)DMS; medium+50 μM R(−)DMS; medium+0.5 μM S(+)DMS; medium+5 μM S(+)DMS; and medium+50 μM S(+)DMS. All of the cell cultures shown in the figure were exposed to 100 μM NMDA. Statistical. significance was determined by ANOVA followed by Dunnett's test. One star above a bar indicates that uptake differs significantly from control at the 0.05 confidence level. Two stars indicate a result that differs at the 0.01 confidence level.

FIG. 17: Relative Effectiveness of R(−)DMS and S(+)DMS on Survival of Cultured Mesencephalic Cells (NMDA Model). Rat mesencephalic cell cultures were exposed to 100 μM NMDA and incubated as described above in connection with FIG. 16. The effect of DMS enantiomers on the survival of TH positive cells is shown in FIG. 17. The bars are in the same order as for FIG. 16 and results are expressed as a percentage of control. One star indicates p<0.05 and two stars indicates p<0.01 when results are compared to those obtained for cells exposed to NMDA and then incubated in unsupplemented medium.

FIG. 18: Inhibition of Neuronal Dopamine Uptake by Deprenyl and the Two Enantiomers of Desmethylselegiline. An in vitro nerve terminal preparation (synaptosome preparation) was prepared using fresh rat neostriatal tissue. This was examined for its ability to take up tritiated dopamine in buffer alone or in buffer supplemented with various concentrations of selegiline, R(−)desmethylselegiline or S(+)desmethylselegiline. Uptake in the presence of each MAO inhibitor, expressed as a percent inhibition vs. log concentration of inhibitor is shown in FIG. 18. As indicated, the plot was used to determine the IC₅₀ for each test agent.

FIG. 19: Determination of IC₅₀ Values for Inhibition of Dopamine Uptake. The experiment of FIG. 18 was repeated in a concentration range designed to more accurately provide an IC₅₀ value and results are shown in FIG. 19. Using the log C vs. probit graphs, as shown in the figure, the IC₅₀ for S(+)DMS was determined to be about 11 μM; for selegiline, about 46 μM; and for R(−)DMS about 54 μM.

FIG. 20: In vivo MAO-B Inhibition in Guinea Pig Hippocampus. Various doses of selegiline, R(−)-desmethylselegiline, and S(+)-desmethylselegiline were administered daily to guinea pigs for a period of 5 days. Animals were then sacrificed and the MAO-B activity in the hippocampus portion of the brain was determined. Results were expressed as a percent inhibition relative to hippocampus MAO-B activity in control animals and are shown in FIG. 20. The plots were used to estimate the ID50 dosage for each agent. The ID₅₀ for selegiline was about 0.008 mg/kg; for R(−)DMS, it was about 0.2 mg/kg; and for S(+)DMS, it was about 0.5 mg/kg.

### DETAILED DESCRIPTION OF THE INVENTION

In the following description, reference will be made to various methodologies well known to those skilled in the art of medicine and pharmacology. Such methodologies are described in standard reference works setting forth the general principles of these disciplines.

The present disclosure is directed to the treatment of tissue damage secondary to cerebrovascular disease or disorder using R(−)DMS, S(+)DMS, or a combination of R(−)DMS and S(+)DMS. Cerebrovascular disease or disorder is a general description of many disorders or injuries that can cause damage to the brain, often due to abnormalities within cerebral circulation. As used herein, the term “cerebrovascular disease” refers to conditions that result in damage to the brain by cerebral ischemia, stroke, transient ischemic attack, intracranial hemorrhage, occlusive hemorrhage, cerebral hemorrhage, subarachnoid hemorrhage, cerebral hypoxia, hemorrhagic lesion, subdural hematoma, aneurysm, mycotic aneurysm, venous occlusion, diffuse ischemia, cerebral abscess, physical injury, or accident.

The term “ischemia” as used herein refers to local anemia due to a mechanical obstruction (such as arterial narrowing) of the blood supply. The term “hypoxia” as used herein refers to a decrease to below normal levels of oxygen in blood or tissue. The usual pathologic outcome is infarction. As used herein, the term “stroke” refers to a neurologic deficit lasting more than 24 hours, which is caused by reduced blood flow in a particular artery supplying the brain. The term “stroke” can also refer to a transient ischemic attack. The term “transient ischemic attack” (“TIA”) as used herein is defined as a similar neurologic deficit lasting less than 24 hours. Most TIAs resolve within an hour, and if a deficit lasts longer than one hour it is likely to be classified as a presumptive stroke and is often associated with permanent brain injury. A subdural hematoma can be distinguished from a stroke because the hematoma has a more prolonged course, as well as a combination of diffuse and focal dysfunction. The relevant clinical distinction between a stroke and a TIA is whether the ischemia caused brain damage (infarction or selective ischemic necrosis).

Although the brain performs no mechanical work, the energy demands to support normal electrophysiologic brain activity in conscious humans equal, on a per weight basis, those of metabolically active tissues like the heart and kidney. Unlike muscle or other tissues, the brain stores very little energy reserves (e.g., glucose, glycogen, or other high-energy phosphate such as ATP and phosphocreatine), and instead relies on a sizable and well-regulated blood flow to satisfy its immediate needs for energy. Major arteries such as the left and right internal carotid and vertebral arteries help supply the blood flow demanded by the brain. In the absence of such blood flow, the brain has sufficient high-energy stores to support normal metabolic needs for only a few minutes. In severely ischemic brain tissue, energy-rich compounds can be depleted within seconds to a few minutes. In fact, changes in synaptic activity, whether related to thinking, talking, or directing muscular activity, are tightly coupled, both temporally and anatomically, to an almost instantaneous, proportional increase in cerebral blood flow (CBF). A complex system of neural pathways regulates CBF in response to normal and abnormal circumstances. Some of these neural pathways participate in autoregulation, a process that maintains CBF at a constant level despite wide fluctuations in cerebral perfusion pressure.

The pathophysiology and pathology of cerebral ischemia due to reduced blood flow to the brain generates a cascade of events that vary qualitatively and quantitatively with the severity of the insult. The severity of cerebral ischemia, which is determined by the degree and duration of blood flow loss, largely decides whether the brain suffers only temporary dysfunction, irreversible injury to a few highly vulnerable neurons (selective ischemic necrosis), or damage to extensive areas involving all cell types (cerebral infarction). For example, if blood flow is restored within 15 to 30 minutes, and no other complicating variables such as hyperglycemia are involved, many of the events that cause the brain tissue to lose its structural integrity can be reversed, and only selectively vulnerable neurons will die. If ischemia lasts for hours or more, however, cerebral infarction develops. Cerebral infarction is often caused by focal vascular occlusion, and is characterized by necrosis of neurons, glia, and endothelial cells.

In contrast to the rapid cascade of events caused by severe ischemia, moderate ischemia triggers poorly defined mechanisms that sacrifice electrophysiologic activity to preserve brain structure, at least temporarily. An acute reduction of blood flow below one half that of normal will exceed the capacity of compensatory mechanisms of the brain, and will cause the patient to become confused, lethargic, or stuporous. A prompt recovery of blood flow will restore full function and structural integrity to the tissue, but if moderate ischemia persists for several hours, irreversible injury begins to develop.

The clinical manifestations of ischemic stroke can depend on the particular blood vessel that is occluded. For example, occlusion of the internal carotid artery can lead to ipsilateral blindness, while occlusion of the middle cerebral artery can manifest as contralateral hemiparesis, sensory loss, expressive aphasia or anosognosia, spatial disorientation, or contralateral inferior quadrantanopsia; occlusion of the anterior cerebral artery can manifest as contralateral hemiparesis or sensory loss; occlusion of the posterior cerebral artery can manifest as contralateral homonymous hemianopsia, superior quadrantanopsia, or memory impairment; occlusion of the basilar apex can manifest as bilateral blindness or amnesia; occlusion of the basilar artery can manifest as contralateral hemiparesis, sensory loss, or ipsilateral bulbar or cerebellar signs; occlusion of the vertebral artery or the posterior inferior cerebellar artery can manifest as ipsilateral loss of facial sensation, ataxia, contralateral hemiparesis, or sensory loss; and occlusion of the superior cerebellar artery can manifest as gait ataxia, nausea, dizziness, headache progressing to ipsilateral hemiataxia, dysarthria, gaze paresis, contralateral hemiparesis, or somnolence.

Cerebrovascular disease can be caused by a wide range of conditions. For example, atherosclerosis of extracranial and intracranial arteries accounts for approximately two thirds of all ischemic strokes, with a greater proportion affecting those over the age of 60. Atherosclerosis can cause strokes either by in situ stenosis or occlusion or by embolization of plaque thrombus material to distal cerebral vessels. Up to one third of all ischemic strokes are caused by cerebral emboli of a cardiac source. Emboli of cardiac origin may be caused by mural thrombus (for example, myocardial infarction (e.g., anterior wall sputum, akinetic segment) or cardiomyopathy (e.g., infectious, idiopathic)); valvular heart disease (e.g., rheumatic heart disease, bacterial endocarditis, non-bacterial endocarditis, Libman-Sacks disease); mitral valve prolapse; prosthetic valve; arrhythmia; cardiac myxoma; or paradoxical emboli. Atherosclerosis is also associated as a cause of ischemic stroke in Behcet's disease; as well as infectious vasculitis caused by for example neurovascular syphilis, Lyme disease, bacterial and fungal meningitis, tuberculosis, acquired immunodeficiency syndrome (AIDS), ophthalmic zoster, and hepatitis.

Thrombus formation and the release of thromboemboli from the heart are promoted by arrhythmias such as atrial fibrillation and structural abnormalities of the valves and chambers. Infective endocarditis caused by staphylococci, fungi, or yeast is associated with emboli that occlude proximal intracranial arteries, as well as other cerebrovascular disease including cerebral hemorrhage, subarachnoid hemorrhage, and mycotic aneurysm, as well as cerebral abscess. In nonbacterial endocarditis, platelet-fibrin vegetations can form on heart valves and then embolize into the systemic circulation. Other causes of cerebrovascular disease include hematologic abnormalities such as hemoglobinopathies (e.g. sickle cell disease), hyperviscosity syndrome (e.g. associated with polycythemia, thrombocytosis, leukocytosis, macroglobulinemia, or multiple myeloma), hypercoagulable states (e.g. associated with cancer, particular adenocarcinomas, pregnancy, and puerperium), protein C or S deficiency, or antiphospholipid antibodies.

A group of disorders classified as vasculitides can cause focal or multifocal cerebral ischemia through inflammation and necrosis of extracranial and/or intracranial blood vessels. Vasculitides that can result in cerebrovascular disease include but are not limited to primary central nervous system vasculitis, systemic necrotizing vasculitis (e.g., polyarteritis nodosa, allergic angiitis), hypersensitivity vasculitis (e.g., serum sickness, drug-induced, cutaneous vasculitis), collagen vascular diseases (e.g., rheumatoid arthritis, scleroderma, Sjorgren's disease), giant cell (temporal arteritis, Takayasu's arteritis), Wegener's granulomatosis, and Lymphomatoid granulomatosis. While the pathogenesis of vascular inflammation differs among these disorders, all involve some deposition of humoral and cellular immune complexes, as well as infiltration of polymorphonuclear and mononuclear cells in blood vessel walls. Although the cause of the inflammatory response is often unknown, infection, a postinfectious or neoplastic process, or a hypersensitivity immune reaction are known to trigger the inflammation. Segmental inflammation of cerebral blood vessels can also cause cerebral ischemia acutely at the site of involvement through platelet aggregation and/or clot formation, or chronically through fibrinoid necrosis, which narrows the vessel lumen.

Cerebrovascular disease can be diagnosed by taking a history of the subject and performing a physical examination of the subject. Laboratory tests such as hematologic tests (e.g. blood and platelet counts), cardiovascular examinations (e.g. electrocardiogram, stress testing), brain imaging (e.g. computed tomographic (CT) scanning, magnetic resonance imaging (MRI)), and/or lumbar puncture, can also be diagnostic and extremely informative in helping to determine the exact nature of the dysfunction. Several noninvasive techniques can also be used to evaluate the cerebrovascular supply in the subject. For example, indirect tests that can examine blood flow are Doppler sonography and quantitative oculopneumoplethysmography, while direct examination can be done using duplex ultrasonography. The direction and velocity of blood flow in intracranial blood vessels can be examined using low-frequency pulsed transcranial Doppler, CT, or MRI images, or magnetic resonance angiography. Cerebral blood flow can also be measured using cerebral angiography, as well as positron emission tomographic (PET) methods, single-photon emission computed tomography (SPECT), and radiolabeled and stable xenon inhalation techniques that utilize CT imaging.

Treating a subject with cerebral ischemia, occlusive hemorrhage, stroke, hypoxia, transient ischemic attack, hemorrhagic lesion, subderal hematoma, aneurysm, physical injury or accident, by administering R(−)DMS, S(+)DMS, or a mixture thereof, will reduce the severity of the effects of reduced blood flow, brain swelling, and/or increased intracranial pressure by reducing neuronal loss, thereby reducing the damage suffered by the brain during the period of reduced cerebral blood flow. Treating a subject with R(−)DMS, S(+)DMS or a combination thereof may reduce the extent and severity of injury to highly vulnerable neurons, as well as other cell types in the brain. This delay will also increase the effectiveness of other therapies to treat cerebrovascular disease by increasing the amount of time before irreversible brain injury occurs.

Cerebral hypoxia/ischemia that can be treated with R(−)DMS, S(+)DMS, or a combination of the two can be categorized into focal or multifocal ischemia from vascular occlusion; global ischemia from complete failure of cardiovascular pumping; and diffuse hypoperfusion-hypoxia caused by respiratory disease or reduced perfusion pressure. Focal cerebral ischemia, resulting most frequently from embolic or thrombotic occlusion of extracranial or intracranial blood vessels, variably reduces blood flow within the involved vascular territory. Blood flow to the central zone of the ischemic vascular bed is usually severely reduced but rarely reaches zero because of partial filling from collateral blood vessels. In transition zones between normally perfused tissue and the severely ischemic central core, blood flow is moderately reduced. This rim of moderately ischemic tissue has been called the ischemic penumbra, and although brain cells in this region remain viable longer than do those in the ischemic core, they too will die if left deprived of adequate blood flow. The middle cerebral artery (MCA) provides flow to most of the lateral surface of the cerebral hemispheres and is a vessel frequently involved in ischemic stroke.

Focal cerebral ischemia sufficient to cause clinical signs or symptoms and lasting only 15 to 30 minutes can cause irreversible injury to specific, highly vulnerable neurons. If the ischemia lasts an hour or longer, infarction of part or all of the involved vascular territory is inevitable. Neuronal loss due to focal cerebral ischemia can be reduced or alleviated by treating the subject with R(−)DMS, S(+)DMS, or a mixture of the two after the symptoms of cerebral ischemia are recognized.

Global cerebral ischemia, which is typically caused by cardiac asystole or ventricular fibrillation, reduces blood flow to zero throughout the entire brain. Global ischemia lasting more than 5 to 10 minutes is usually incompatible with recovery of consciousness in humans. Brain damage from more transient global ischemia, uncomplicated by periods of prolonged hypotension or hyperglycemia, is limited to specific populations of highly vulnerable neurons, causing selective ischemic necrosis. Selective ischemic necrosis of neurons can evolve more slowly and sometimes takes several days or more to reach its full extent. This selective ischemic necrosis of neurons involves, for example, the CAl pyramidal neurons of hippocampus, the cerebellar Purkinje cells, and the pyramidal neurons in neocortical layers 3, 5, and 6. While selective ischemic necrosis of neurons typifies transient global ischemia, such injury may also accompany prolonged hypoxemia, carbon monoxide poisoning, and focal cerebral ischemia of brief duration. Cardiac resuscitation complicated by prolonged hypotension or hyperglycemia may cause cerebral infarction, particularly in border zones that lie between the terminal branches of major arterial supplies. Again, the severity of global cerebral ischemia can be reduced or alleviated by treatment with R(−)DMS, S(+)DMS, or a combination thereof.

Cerebral edema, a pathologic increase in the water content of the brain that accompanies all types of ischemic and hemorrhagic stroke, may also be treated with R(−)DMS, S(+)DMS, or a combination of the two. Brain swelling and raised intracranial pressure relate proportionally to the volume of the accumulated water, and in some instances can cause neurologic deterioration and death by transtentorial herniation. Cerebral edema, which is categorized as intracellular or interstitial, as well as herniation, are the immediate cause of death in one third of all ischemic and three quarters of all hemorrhagic fatal strokes.

Intracellular edema, also called cytotoxic edema, represents an accumulation of intracellular osmoles and water that cause cell swelling at the expense of the interstitial brain volume, and may also be treated with R(−)DMS, S(+)DMS, or a combination of the two. Intracellular edema develops rapidly in ischemic brain tissue as energy-dependent membrane ion pumps fail and Na⁺ and other osmoles enter the cell from the interstitialand vascular compartments. Although cell swelling occurs predominantly in astrocytes, neurons, and oligodendroglial cells, endothelial cells are also involved to a lesser extent. If cerebral circulation is re-established before permanent brain injury develops, intracellular brain edema resolves within a matter of hours without permanent damage.

Interstitial brain edema, which is also called vasogenic edema, occurs later than the intracellular form of brain edema, and may also be treated with R(−)DMS, S(+)DMS, or a combination of the two. Damage to blood-brain barrier endothelial cells allows macromolecules such as plasma proteins to enter the interstitial space, carrying with them osmotically bound water. Interstitial brain edema that follows cerebral infarction will progressively worsen for 3 or 4 days after a stroke. Fluid accumulation within the vicinity of damaged endothelial cells and the zone of infarction can raise the local water content of brain by as much as 10%, which can lead to transtentorial herniation and other fatal consequences.

Drug-related causes of stroke can also be treated with R(−)DMS, S(+)DMS or a combination of the two. A large number of “street drugs” have been associated with stroke, including but not limited to cocaine, crack, amphetamines, lysergic acid, phencyclidine, methylphenidate, sympathomimetics, heroin, and pentazocine. Stroke may also be caused by sharing non-sterile needles to intravenously inject drugs by precipitating infectious processes (e.g., bacterial endocarditis, hepatitis B, and mycotic aneurysms). Alcohol is also considered a drug related cause of stroke. Some over-the-counter cold remedies, nasal decongestants, and appetite suppressants containing sympathomimetics have also been associated with ischemic stroke. An increased risk for ischemic or hemorrhagic stroke has also been shown among users of high dose estrogen contraceptives.

Other causes of stroke include, but are not limited to, fibromuscular dysplasia (or hyperplasia), arterial dissection (trauma, spontaneous, Marfan syndrome), homocystinuria, migraine, subarachnoid hemorrhage or vasospasm (reactive vascular narrowing), emboli (fat, bone marrow, air), and moyamoya.

Approximately 20% of strokes are caused by intracranial hemorrhages, about half of which hemorrhage into brain tissue (parenchymatous hemorrhage), while the other half hemorrhage into the surrounding subarachnoid space (subarachnoid hemorrhage). In hemorrhagic cerebrovascular disease, an arterial rupture can lead to an acute rise in intracranial pressure, which causes loss of consciousness in about half of patients, many of which die of cerebral herniation. Nevertheless, because hemorrhage into the subarachnoid space or brain parenchyma can cause less tissue injury than ischemia, patients who survive often have a remarkable recovery. Clinically, intracranial hemorrhages may present as a putaminal hemorrhage, thalamic hemorrhage, pontine hemorrhage, cerebellar hemorrhage, or lobar cerebral hemorrhage. There are many causes of spontaneous intracranial hemorrhages, including but not limited to arterial aneurysms such as saccular or “Berry” aneurysm, fusiform aneurysm, mycotic aneurysm, and aneurysm with vasculitis; cerebrovascular malformations; hypertensive-atherosclerotic hemorrhage; hemorrhage into brain tumor; systemic bleeding diatheses; hemorrhage with vasculopathies; and hemorrhage with intracranial venous infarction.

Hemorrhagic strokes can be categorized either as diffuse (subarachnoid or intraventricular) or focal (intraparenchymal). Subarachnoid hemorrhage can be caused by the rupture of surface arteries (aneurysms, vascular malformations, head trauma), usually limited to the cerebrospinal fluid space between the pial and arachnoid membranes. While intracerebral hemorrhage is most frequently caused by the rupture of arteries lying deep within the brain substance (hypertensive hemorrhage, vascular malformations, head trauma), in some instances the force of blood from ruptured surface arteries can penetrate the brain parenchyma. Focal hemorrhages often occur spontaneously in three common settings: hypertension, ruptured arteriovenous malformations, and amyloid (or congophilic) angiopathy; other contributing factors are excessive anticoagulation, fibrinolysis, hematologic abnormalities, systemic bleeding diatheses, and trauma. Cerebral hemorrhages can also occur in patients with leukemia, polycythemia, hemophilia, and other clotting abnormalities, as well as in patients who use amphetamines, cocaine, and other sympathomimetics. Congenital, acquired, and heredity are all factors that can contribute to the pathogenesis of hemorrhagic stroke. There are five general categories of congenital vascular malformations of the brain and spinal cord: venous angiomas, cerebral varix, telangiectasias, cavernous angiomas, and arteriovenous malformation.

The objective of the present disclosure is to administer R(−)DMS, S(+)DMS, or a racemic mixture of R(−)DMS and S(+)DMS to prevent, treat, reduce, or eliminate the symptoms and damage associated with cerebrovascular disease. R(−)DMS, S(+)DMS, or a combination of R(−)DMS and S(+)DMS may also be administered prophylactically. The particular cerebrovascular disease is not critical to the present disclosure. Thus, R(−)DMS, S(+)DMS, or a mixture of R(−)DMS and S(+)DMS, is effective for treating damage caused by cerebrovascular diseases associated with ischemia, stroke, transient ischemic attack, intracranial hemorrhage, occlusive hemorrhage, cerebral hemorrhage, subarachnoid hemorrhage, hypoxia, hemorrhagic lesion, subderal hematoma, aneurysm, mycotic aneurysm, venous occlusion, diffuse ischemia, cerebral abscess, physical injury, or accident.

Without being bound by any particular theory, R(−)DMS, S(+)DMS, or a mixture thereof may treat or prevent symptoms and damage associated with cerebrovascular disease in part by reducing neuronal loss due to apoptosis that would otherwise result from reduced cerebral blood flow, brain swelling, and/or increased intracranial pressure. Apoptosis is a process of cell suicide that is characterized morphologically by cell shrinkage, chromatin aggregation with extensive genomic fragmentation, and nuclear pyknosis. Excessive apoptosis has been associated with cerebrovascular diseases such as ischemic stroke and hypoxia. Thus, the molecular mechanisms of apoptosis can be activated in neuropathological states such as, for example, stroke, cerebral ischemia, hypoxia, or traumatic brain injury. Using one or both of the enantiomers of desmethylselegiline to reduce neuronal apoptosis may result in a reduction of the extent and severity of injury to highly vulnerable neurons, as well as other cell types in the brain, thus slowing the progression of and/or damage caused by a cerebrovascular disease.

Presently there are few generally accepted therapies for treatment of the consequences of cerebrovascular disease. Patients who present within 3 hours of ischemic stroke onset may be treated with intravenous thrombolytic therapy. Tissue plasminogen activator (tPA) in a dose of 0.9 mg/kg (maximum dose 90 mg) can be administered intravenously, with 10% given as a bolus and the remaining 90% by infusion over 60 minutes. Aspirin (325 mg) may be given to patients within 24 hours of symptom onset who are not treated with tPA, or after 24 hours to those who are treated with tPA. Anticoagulant therapy with intravenous heparin, low-molecular-weight heparins, or heparinoids may also be used to therapeutically treat patients with certain cerebrovascular diseases such as atherothrombosis of large intracranial or extracranial arteries. Aneurysms such as saccular aneurysms can be treated by surgical clipping of the aneurysm to prevent rebleeding. Focused radiation therapy (e.g., focused gamma x-rays or proton beam radiation) and surgery can also be used in conjunction with arterial embolization to treat hemorrhagic cerebrovascular diseases. Nimodipine, a voltage-regulated calcium channel antagonist, which can be given orally, has been shown to lower by one third the incidence of cerebral infarction in patients suffering from subarachnoid hemorrhage and cerebral vasospasm. Phenylephrine or dopamine can also be administered to partly overcome the effects of cerebral vasospasm by raising cerebral perfusion pressure through plasma volume expansion.

R(−)DMS, S(+)DMS, or a racemic mixture of R(−)DMS and S(+)DMS may be administered prior to, concurrently with, in combination with, or subsequent to the administration of other therapies for cerebrovascular disease such as aspirin; tPA; heparin; low-molecular-weight heparins; heparinoids; ticlopidine; clopidogrel;, warfarin; glutamate receptor antagonists; sodium, potassium, and channel blockers; antioxidants; anti-inflammatory compounds; nimodipine; phenylephrine; dopamine; or growth factors, to prevent, treat, reduce, or eliminate the symptoms associated with the cerebrovascular disease. R(−)DMS, S(+)DMS, or a racemic mixture of R(−)DMS and S(+)DMS can also be administered prior to, concurrently, or subsequent to surgical therapies, e.g. surgical clippings of aneurysms, or focused radiation therapies for the treatment of cerebrovascular diseases such as hemorrhagic cerebrovascular disease.

The present disclosure further encompasses methods for treating cerebrovascular disease by administering to the patient a pharmaceutical composition that includes R(−)DMS, S(+)DMS, or combinations of the two (which are conveniently prepared by methods known in the art, as described in Example 1) and one or more additional therapeutic agents that may treat cerebrovascular disease, for example aspirin; tPA; heparin; low-molecular-weight heparins; heparinoids; ticlopidine; clopidogrel;, warfarin; glutamate receptor antagonists; sodium, potassium, and channel blockers; antioxidants; anti-inflammatory compounds; nimodipine; phenylephrine; dopamine; or growth factors. Such a pharmaceutical composition may be used to prevent or treat cerebrovascular disease. The therapeutic agents used in combination with R(−)DMS, S(+)DMS, or a mixture of the two to prevent or treat cerebrovascular disease can be presented to the patient in separate formulations. Thus, separate administration of a therapeutic agent or even administration that is spaced in time is contemplated by the present disclosure, particularly when the therapeutic agent and the DMS enantiomer or DMS enantiomers have a synergistic therapeutic action.

As stated, the present disclosure encompasses the treatment of neurosystem damage associated with cerebrovascular disease, including the prevention, alleviation, reduction, or elimination, in whole or in part, of symptoms associated with a cerebrovascular disease, by use of DMS in the form of R(−)DMS, S(+)DMS, or mixtures of R(−)DMS and S(+)DMS. As used herein, the term R(−)DMS means the R(−) enantiomeric form of DMS, including as a free base, as well as any acid addition salt thereof. Likewise, the term S(+)DMS, as used herein, encompasses the S(+) enantiomeric form of DMS, including as a free base, as well as any acid addition salt thereof. Such salts of either R(−)DMS or S(+)DMS include those derived from organic and inorganic acids such as, without limitation, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embonic acid, enanthic acid, and the like. Accordingly, reference herein to the administration of either or both R(−)DMS and S(+)DMS encompasses both the free base and acid addition salt forms. When either R(−)DMS or S(+)DMS is used alone in the presently disclosed compositions and methods, it is used in a substantially enantiomerically pure form. Reference to mixtures or combinations of R(−)DMS and S(+)DMS includes both racemic and non-racemic mixtures of optical isomers.

R(−)DMS and/or S(+)DMS may be administered either by an oral route (involving gastrointestinal absorption) or by a non-oral route (does not rely upon gastrointestinal absorption, i.e. a route that avoids absorption of R(−)DMS and/or S(+)DMS from the gastrointestinal tract). Depending upon the particular route employed, the DMS is administered in the form of the free base or as a physiologically acceptable non-toxic acid addition salt as described above. The use of salts, especially the hydrochloride, is particularly desirable when the route of administration employs aqueous solutions, as for example parenteral administration; use of delivered desmethylselegiline in the form of the free base is especially useful for transdermal administration. Although the oral route of administration will generally be most convenient, R(−)DMS, S(+)DMS, or a mixture of both may be administered by oral, peroral, enteral, pulmonary, nasal, lingual, intravenous, intraarterial, intracardial, intramuscular, intraperitoneal, intracutaneous, subcutaneous, parenteral, topical, transdermal, intraocular, buccal, sublingual, intranasal, inhalation, vaginal, rectal, or other routes as well.

The optimal daily dose of R(−)DMS, S(+)DMS, or of a combination of the two, such as a racemic mixture of R(−)DMS and S(+)DMS, useful for the purposes of the present disclosure is determined by methods known in the art, e.g., based on the severity of the cerebrovascular disease and symptoms being treated, the condition of the subject to whom treatment is being given, the desired degree of therapeutic response, and the concomitant therapies being administered to the patient or animal. The total daily dosage administered to a patient, typically a human patient, should be at least the amount required to prevent, reduce, or eliminate one or more of the symptoms associated with cerebrovascular disease, typically one of the symptoms discussed herein. Prophylactic administration of R(−)DMS and/or S(+)DMS to a patient may also be used to prevent or reduce damage caused by cerebrovascular disease.

Ordinarily, the attending physician will administer an initial daily non-oral dose of at least about 0.01 mg per kg of body weight, calculated on the basis of the free secondary amine, with progressively higher doses being employed depending upon the response to therapy. The final daily dose will be between about 0.05 mg/kg of body weight and about 0.15 mg/kg of body weight (all such doses again being calculated on the basis of the free secondary amine). Ordinarily, however, the attending physician or veterinarian will administer an initial dose of at least about 0.015 mg/kg, calculated on the basis of the free secondary amine, with progressively higher doses being employed depending upon the route of administration and the subsequent response to the therapy. Typically the daily dose will be from about 0.02 mg/kg or 0.05 mg/kg to about 0.10 mg/kg or about 0.15 mg/kg to about 0.175 mg/kg or about 0.20 mg/kg or about 0.5 mg/kg and may extend to about 1.0 mg/kg or even 1.5, 2.0, 3.0 or 5.0 mg/kg of the patient's body weight depending on the route of administration. Preferred daily doses will be in the range of about 0.10 mg/kg to about 1.0 mg/kg. More preferred daily doses will be in the range of about 0.4 mg/kg to about 0.9 mg/kg. Even more preferred daily doses will be in the range of about 0.6 mg/kg to about 0.8 mg/kg. In other preferred embodiments, the daily dose will be in the range of about 0.01 mg to about 1000 mg per day. Preferred doses will be about 0.05, 0.1, 0.2, 0.3, 0.4, 0.5, 1.0, 2.0, 3.0, 4.0, 5.0, 10, 20, 30, 40, 50, 60, 70, 80, 90, 100, 150, 200, 300, 400, 500, 600, 700, 800, 900, or 1000 mg per day. Again, all such doses should be calculated on the basis of the free secondary amine.

In instances in which a subject is taking multiple drugs or in which there is some reason to believe that they may be unusually sensitive to R(−)DMS, S(+)DMS, or a combination of the two, it may be desirable to start with a low initial dose (e.g., 0.01 mg/kg) in order to ensure that the subject is able to tolerate the medication. Once this is established, the dosage may be adjusted upward. The effect of R(−)DMS, S(+)DMS, or a combination of the two on the symptoms of cerebrovascular disease should be evaluated by the subject over a period of time and by the subject's physician on a regular basis.

These are simply guidelines since the actual dose must be carefully selected and titrated by the attending physician based upon clinical conditions. The optimal daily dose will be determined by methods known in the art and will be influenced by factors such as the age and weight of the patient, the clinical condition of the patient, the cerebrovascular disease presented by the patient, the severity of the damage related to cerebrovascular disease, the symptoms demonstrated, the condition of the patient to whom treatment is being given, the desired degree of therapeutic response, the concomitant therapies being administered, and observed response of the individual patient or animal. The daily dose can be administered in a single or multiple dosage regimen. The particular route of administration of R(−)DMS, S(+)DMS, or a mixture of R(−)DMS and S(+)DMS that is most preferred for a patient treated for a particular cerebrovascular disease will be determined by clinical considerations and may include any of the routes of delivery or dosage forms discussed above. Routes of administration which avoid gastrointestinal absorption may be preferred. Thus, preferred routes will typically include transdermal, intravenous, intra-arterial, sublingual, buccal, or other parenteral routes of administration.

Either oral or non-oral dosage forms may be used and may permit, for example, a burst of the active ingredient from a single dosage unit, such as an oral composition, intravenous, sublingual or buccal administration, or a continuous release of relatively small amounts of the active ingredient from a single dosage unit, such as a transdermal patch or intravenous infusion, over the course of one or more days. Alternatively, intravenous or inhalation routes may be preferred. A number of different dosage forms may be used to administer the R(−)DMS, S(+)DMS, or a combination of R(−)DMS and S(+)DMS, including but not limited to tablets, pills, capsules, powders, aerosols, suppositories, skin patches, parenterals, and oral liquids, including oil or aqueous injectable suspensions, solutions, and emulsions. Additionally, desmethylselegiline-containing sustained release (long acting) formulations and devices are contemplated.

Pharmaceutical compositions containing one or both R(−)DMS or S(+)DMS can be prepared according to conventional techniques. For example, preparations for parenteral routes of administration, e.g., intramuscular, intravenous, intrathecal, and intraarterial routes, can employ sterile isotonic saline solutions. Sterile buffered solutions can also be employed for intraocular administration.

Transdermal dosage unit forms of R(−)DMS and/or S(+)DMS can be prepared utilizing a variety of previously described techniques (see e.g., U.S. Pat. Nos. 4,861,800; 4,868,218; 5,128,145; 5,190,763; and 5,242,950; and EP-A 404807, EP-A 509761, and EP-A 593807, incorporated herein by reference). For example, a monolithic patch structure can be utilized in which desmethylselegiline is directly incorporated into the adhesive and this mixture is cast onto a backing sheet. Alternatively R(−)DMS and/or S(+)DMS, can be incorporated as an acid addition salt into a multilayer patch which effects a conversion of the salt to the free base, as described for example in EP-A 593807 (incorporated herein by reference). Specifically contemplated by the present disclosure is a transdermal patch composition that has about 5 mg, 10 mg, 15 mg, 20 mg, 25 mg, 30 mg, 40 mg, 50 mg, or 100 mg of R(−)DMS, S(+)DMS, or a combination of R(−)DMS and S(+)DMS.

One or both R(−)DMS or S(+)DMS can also be administered by a device employing a lyotropic liquid crystalline composition in which, for example, 5 to 15% of desmethylselegiline is combined with a mixture of liquid and solid polyethylene glycols, a polymer, and a nonionic surfactant, optionally with the addition of propylene glycol and an emulsifying agent. For further details on the preparation of such transdermal preparations, reference can be made to EP-A 5509761 (incorporated herein by reference). Additionally, buccal and sublingual dosage forms of R(−)DMS, S(+)DMS, or a combination of R(−)DMS and S(+)DMS may be prepared utilizing techniques described in, for example, U.S. Pat. Nos. 5,192,550; 5,221,536; 5,266,332; 5,057,321; 5,446,070; 4,826,875; 5,304,379; or 5,354,885 (incorporated herein by reference).

Subjects treatable by the present preparations and methods include both human and non-human subjects. Accordingly, the compositions and methods above provide especially useful therapies for mammals, including humans, and domesticated mammals. Thus, the present methods and compositions are used in treating neurosystem damage resulting from cerebrovascular diseases in human, primate, canine, feline, bovine, equine, ovine, murine, caprine, and porcine species, and the like.

Treatment by the administration of R(−)DMS, S(+)DMS, or a combination of R(−)DMS and S(+)DMS should be continued until the symptoms associated with the cerebrovascular disease subside. The drug may be either administered at regular intervals (e.g., twice a day) or delivered in an essentially continuous manner, e.g., via a transdermal patch or intravenous infusion. Patients should be regularly evaluated by physicians, e.g. once an hour, once a day, once a week, once a month, etc., to determine whether there has been an improvement in symptoms and whether the dosage of desmethylselegiline needs to be adjusted. Since irreversible neuronal death is often associated with cerebrovascular disease, R(−)DMS, S(+)DMS, or a mixture of the two is preferably administered immediately to a patient presenting with a cerebrovascular disease, with routine evaluation by a physician to determine whether to continue therapeutic administration of desmethylselegiline. Additionally, the administration of R(−)DMS, S(+)DMS, or a combination of the two maybe used prophylactically to prevent or reduce neuronal damage associated with cerebrovascular disease.

Successful use of the compositions and methods above requires employment of a therapeutically effective amount of R(−)DMS, S(+)DMS, or combination of R(−)DMS and S(+)DMS. As described above and notwithstanding its demonstrably inferior inhibitory properties with respect to MAO-B inhibition, R(−)DMS and its enantiomer appear to be at least if not more effective than selegiline for treating neuronal damage associated with cerebrovascular disease.

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. The following working examples are illustrative only, and are not intended to limit the scope of the invention.

### EXAMPLE 1

### Preparation of R(−)DMS and S(+)DMS

A. R(−)-desmethylselegiline

R(−)DMS is prepared by methods known in the art. For example, desmethylselegiline is a known chemical intermediate for the preparation of selegiline as described in U.S. Pat. No. 4,925,878. Desmethylselegiline can be prepared by treating a solution of R(−)-2-aminophenylpropane (levoamphetamine): 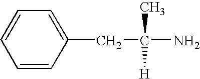
in an inert organic solvent such as toluene with an equimolar amount of a reactive propargyl halide such as propargyl bromide, Br—CH₂—C≡CH, at slightly elevated temperatures (70°-90° C.). Optionally the reaction can be conducted in the presence of an acid acceptor such as potassium carbonate. The reaction mixture is then extracted with aqueous acid, for example 5% hydrochloric acid, and the extracts are rendered alkaline. The nonaqueous layer which forms is separated, for example, by extraction with benzene, dried, and distilled under reduced pressure.

Alternatively the propargylation can be conducted in a two-phase system of a water-immiscible solvent and aqueous alkali, utilizing a salt of R(+)-2-aminophenylpropane with a weak acid such as the tartrate, analogously to the preparation of selegiline as described in U.S. Pat. No. 4,564,706.

B. S(+)-desmethylselegiline

S(+)DMS is conveniently prepared from the enantiomeric S(+)-2-aminophenylpropane (dextroamphetamine), i.e., 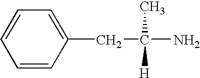
following the procedures set forth above for desmethylselegiline.

C. Mixtures of Enantiomers

Mixtures of the R(−) and S(+) enantiomeric forms of desmethylselegiline, including racemic desmethylselegiline, are conveniently prepared from enantiomeric mixtures, including racemic mixtures of the above aminophenylpropane starting material.

D. Conversion Into Acid Addition Salts

N-(prop-2-ynyl)-2-aminophenylpropane in either optically active or racemic form can be converted to a physiologically acceptable non-toxic acid addition salt by conventional techniques such as treatment with a mineral acid. For example, hydrogen chloride in isopropanol is employed in the preparation of desmethylselegiline hydrochloride. Either the free base or salt can be further purified, again by conventional techniques such as recrystallization or chromatography.

### EXAMPLE 2

### Characteristics of Substantially Pure R(−)DMS

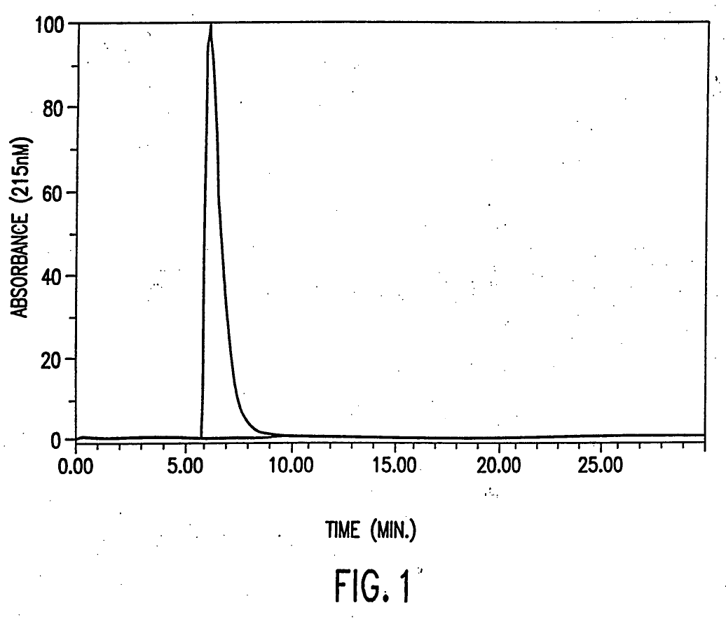

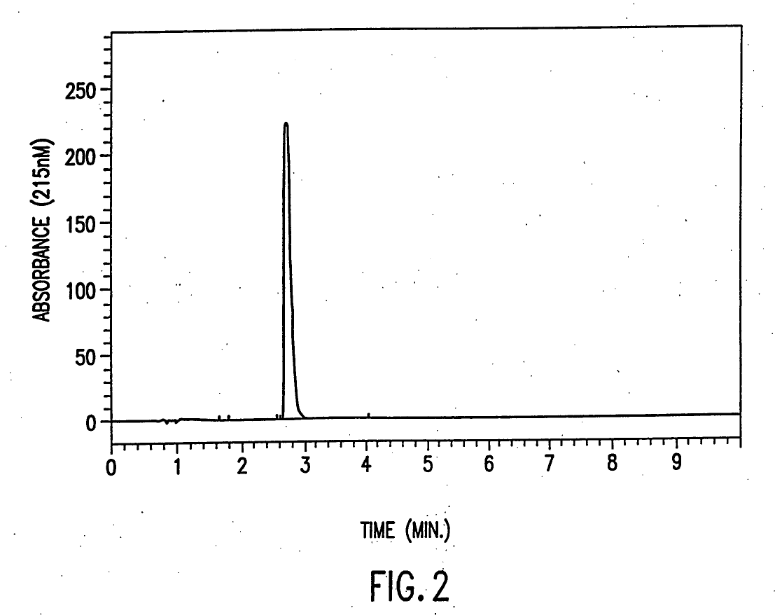

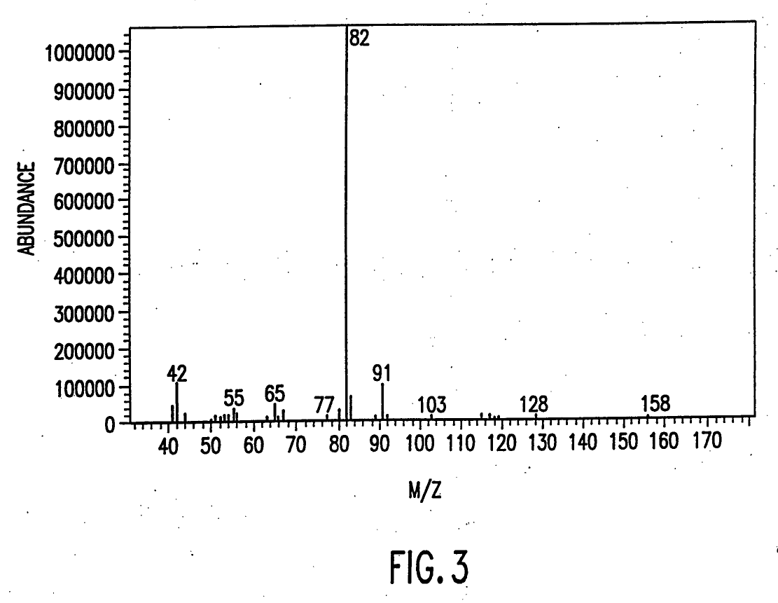

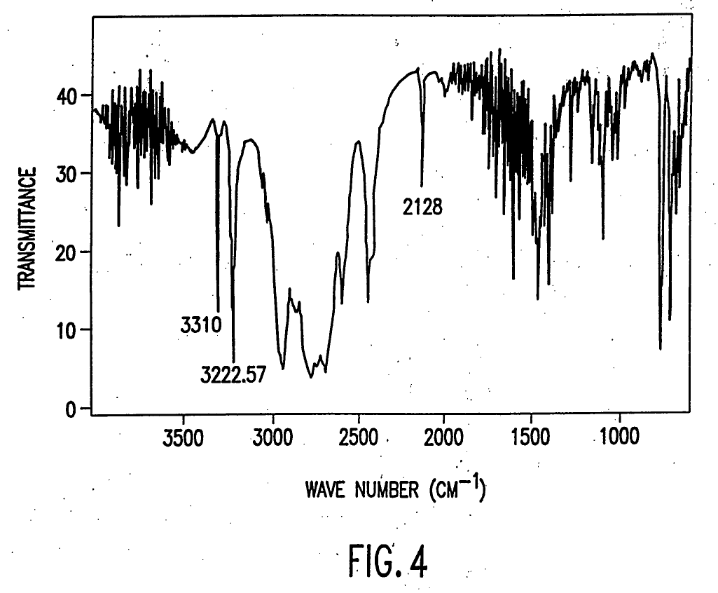

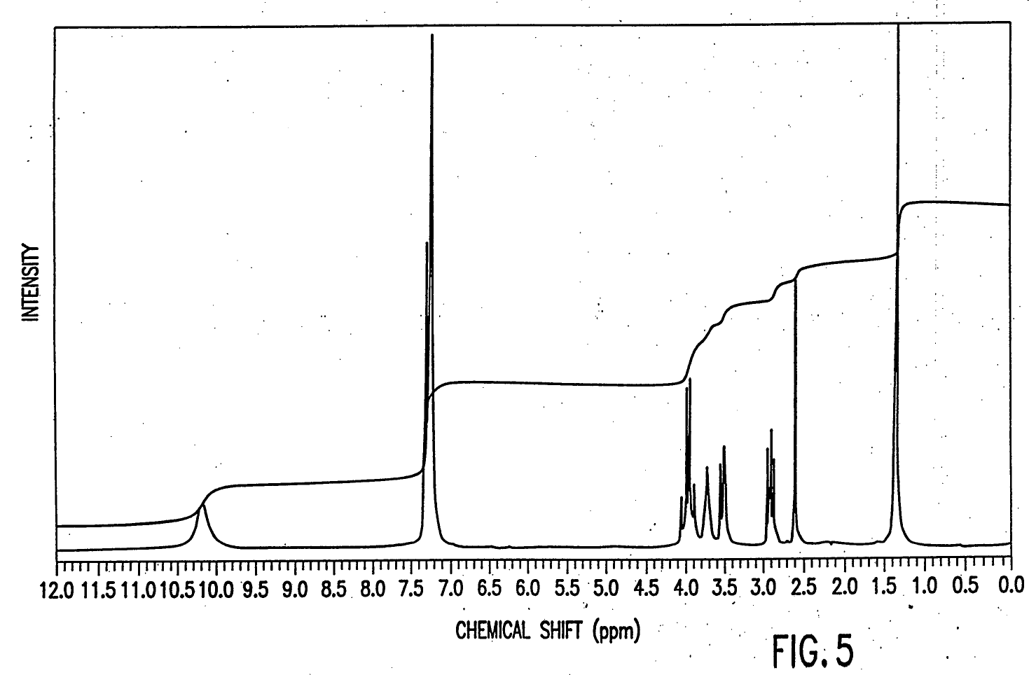

A preparation of substantially pure R(−)DMS has the appearance of a white crystalline solid with a melting point of 162-163 C. and an optical rotation of [α]_{D}^{23c}=−15.2±2.0 when measured at a concentration of 1.0 M using water as solvent. R(−)DMS appeared to be 99.5% pure when analyzed by HPLC on a Microsorb MV Cyano column (see chromatogram in FIG. 1) and 99.6% pure when analyzed by HPLC on a Zorbax Mac-Mod SB-C 18 column, (see chromatogram in FIG. 2). No single impurity is present at a concentration greater than or equal to 0.5%. Heavy metals are present at a concentration of less than 10 ppm and amphetamine hydrochloride at a concentration of less than 0.03%. The last solvents used for dissolving the preparation, ethyl acetate and ethanol are both present at a concentration of less than 0.1%. A mass spectrum performed on the preparation (see FIG. 3) is consistent with a compound having a molecular weight of 209.72 amu and a formula of C₁₂H₁₅N.HCl. Infrared and NMR spectra are shown in FIGS. 4 and 5 respectively. These are also consistent with the known structure of R-(−)-DMS.

### EXAMPLE 3

### Characteristics of Substantially Pure S(+)DMS

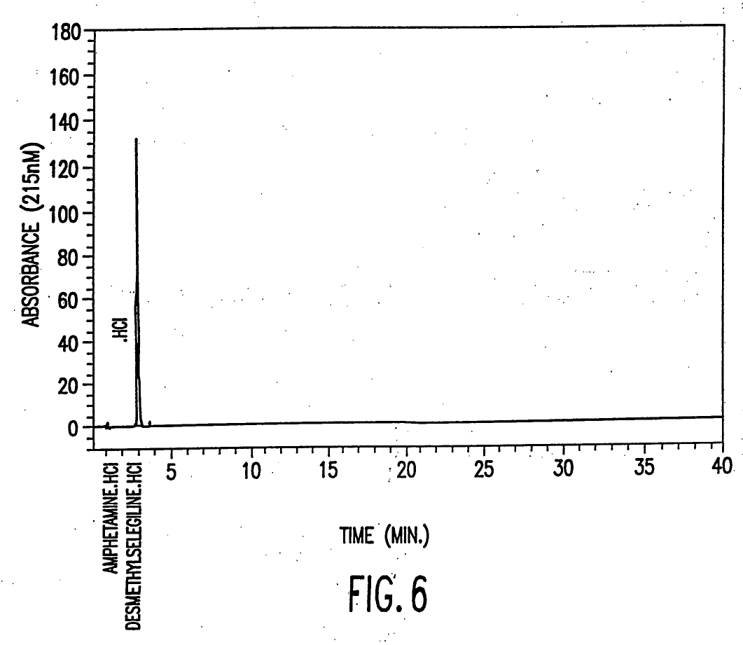

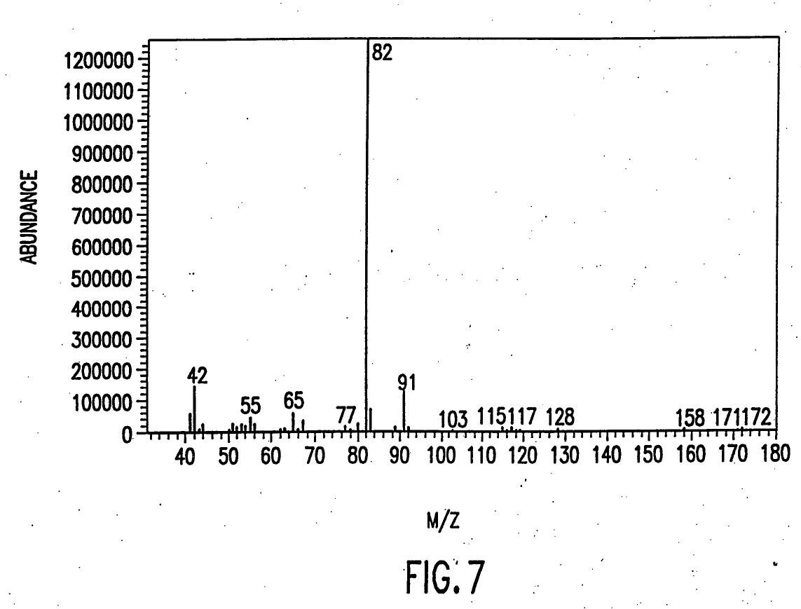

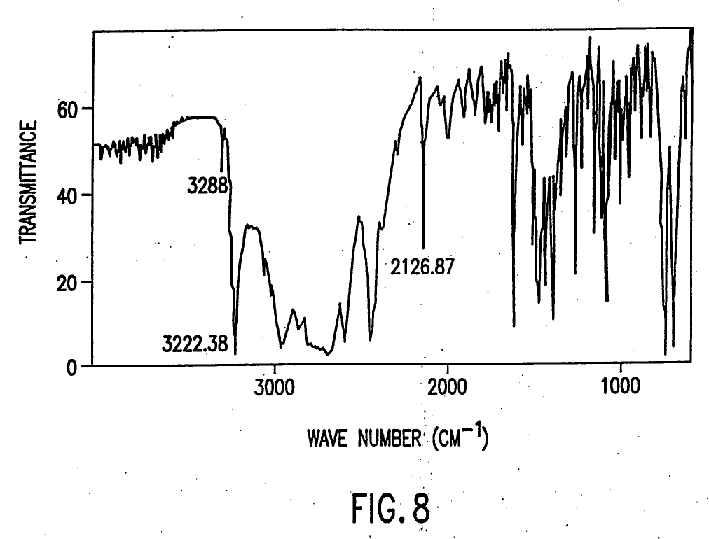

A preparation of substantially pure S(+)DMS has the appearance of a white powder with a melting point of approximately 160.04° C. and a specific rotation of +15.1 degrees when measured at 22° C. in water, at a concentration of 1.0 M. When examined by reverse phase HPLC on a Zorbax Mac-Mod SB-C18 column the preparation appears to be about 99.9% pure (FIG. 6). Amphetamine hydrochloride is present at a concentration of less than 0.13% (w/w). A mass spectrum is performed on the preparation and is consistent with a compound having a molecular weight of 209.72 and a molecular formula of C₁₂H₁₅N.HCI(see FIG. 7). Infrared spectroscopy is performed and also provides results consistent with the structure of S(+)DMS (see FIG. 8).

### EXAMPLE 4

### Neuronal Survival as Measured Using Tyrosine Hydroxylase

The effect of desmethylselegiline on neuron survival can be correlated to tyrosine hydroxylase, the rate limiting enzyme in dopamine biosynthesis. Assays are performed by determining the number of tyrosine hydroxylase positive cells in cultured E-14 embryonic mesencephalic cells over a period of 7 to 14 days. Protection in this system has been seen with a variety of trophic factors including BDNF, GDNF, EGF, and (3-FGF.

A. Test Methods

Timed pregnant Sprague-Dawley rats are used to establish neuronal cultures from embryonic rat brain on the 14th day of gestation. Mesencephalon is dissected out without the membrane coverings and collected in Ca++ and Mg++ free balanced salt solution at 4° C. Tissue fragments are dissociated in chemically defined medium by mild trituration with a small bore pasteur pipette. Cell suspension is plated in polyornithine-coated 35 mm Falcon plastic dishes (0.1 mg/ml, Sigma) at a density of 1.5×10⁶ cells/dish. Cultures are maintained at 37° C. in an atmosphere of 10% C02/90% air and 100% relative humidity, and fed twice weekly with chemically defined medium consisting of MEM/F12 (1:1, Gibco), glucose (33 mM), HEPES (15 mM), NaHC0₃ (44.6 mM), transferrin (100 mg/ml), insulin (25 mg/ml), putrescine (60 nM), sodium selenite (30 nM), progesterone (20 nM), and glutamine (2 mM). Control cells receive no further additions. The medium used for other cells also included test substance, e.g. selegiline or desmethylselegiline, at one or more concentrations.

Cultures are fixed in 4% paraformaldehyde in 0.1 M phosphate buffer (pH 7.4) for 30 minutes at room temperature, permeabilized with 0.2% Triton X-100 for 30 minutes and incubated with an antibody against tyrosine hydroxylase (1:1000; Eugene Tech) for 48 hours at 4° C. in the presence of a blocking serum. They are then stained using a peroxidase-coupled avidin-biotin staining kit (Vectastain ABC kit; Vector Labs) with 3′,3′-diaminobenzidine as a chromagen.

The number of dopaminergic neurons in cultures is determined by counting the cells positively immunostained with TH antibodies. 100 fields (0.5 mm×0.5 mm) in two transverse strips across the diameter of the dish, representing 2.5% of the total area, are counted using a Nikon inverted microscope at 200× magnification.

B. Results

Using the procedures described above, the following results were obtained:

TABLE 2
Effect of Selegiline and DMS on the Survival of TH Positive Cells

| | Control | Selegiline | | Desmethylselegiline | |
|---|---|---|---|---|---|
| | Mean | Mean | | Mean | |
| Conc. | cells/cm² | cells/cm² | % cont. | cells/cm² | % cont. |
| 0.5 μM | 108.55 | 201.70 ± 25.01 | 185.81 | 246.00 ± 22.76 | 226.62 |
| 5 μM | — | 237.00 ± 12.59 | 218.33 | 357.95 ± 25.76 | 329.76 |
| 50 μM | — | 292.28 ± 17.41 | 269.25 | 391.60 ± 34.93 | 360.76 |

### EXAMPLE 5

### Neuronal Survival as Measured Using Dopamine Uptake

In addition to determining the number of TH positive cells in culture (see Example 4) the protective effect of selegiline or desmethylselegiline on neuronal cells also can be determined by directly measuring dopamine uptake. The amount of uptake by the cultured brain cells corresponds to neuronal survival and axonal growth.

A. Test Methods

Cell cultures, established in the manner discussed above, are incubated with [³H]dopamine (0.5 mCi/ml; 37 Ci/mmol; New England Nuclear) for 15 minutes in the presence of ascorbic acid (0.2 mg/ml) in PBS (pH 7.3), supplemented with 0.9 mM CaCl₂ and 0.5 mM MgCl₂ at 37° C. After two rinses and a 5 minute incubation with fresh buffer, [³H]dopamine accumulated within the cells is released by incubating the cultures with 95% ethanol for 30 minutes at 37° C. Preparations are then added to 10 ml Ecoscint (National Diagnostics) and counted in a scintillation spectrometer. Nonspecific uptake values are obtained by blocking dopaminergic neuronal uptake with 10 mM mazindol.

B. Results

Using the above procedure, the results shown in Table 3 were obtained.

TABLE 3
Effect of Selegiline and DMS on ³H-Dopamine Uptake

| | Control | Selegiline | | Desmethylselegiline | |
|---|---|---|---|---|---|
| Conc. | Mean | Mean | % Cont. | Mean | % Cont. |
| 0.5 μM | 11982 | 14452 ± 212 | 120.6 | 24020 ± 800 | 200.4 |
| 5 μM | — | 16468 ± 576 | 137.5 | 34936 ± 2119 | 291.5 |
| 50 μM | — | 33018 ± 1317 | 275.5 | 56826 ± 2656 | 474.3 |

C. Conclusions from Examples 4 and 5

The results described in Examples 4 and 5 indicate that desmethylselegiline is substantially more potent compared to selegiline as a neuroprotective agent. This is true notwithstanding the fact that desmethylselegiline in much less potent than selegiline as an inhibitor of MAO-B.

### EXAMPLE 6

### Neuroprotective Action of Desmethylselegiline Enantiomers in Cultured Dopamine-Containing Mesencephalic Neurons In Vitro

The survival of mesencephalic, dopamine-containing neuronal cultures of rat brain tissue was used in these experiments to examine neuroprotective properties of selegiline or R(−) desmethylselegiline. The number of TH positive neurons is directly proportional to the survival of dopaminergic neurons and ³H-dopamine uptake is a measure of neuronal survival and axonal growth in these neurons

A. Effect of Selegiline on the Survival of Dopaminergic Neurons.

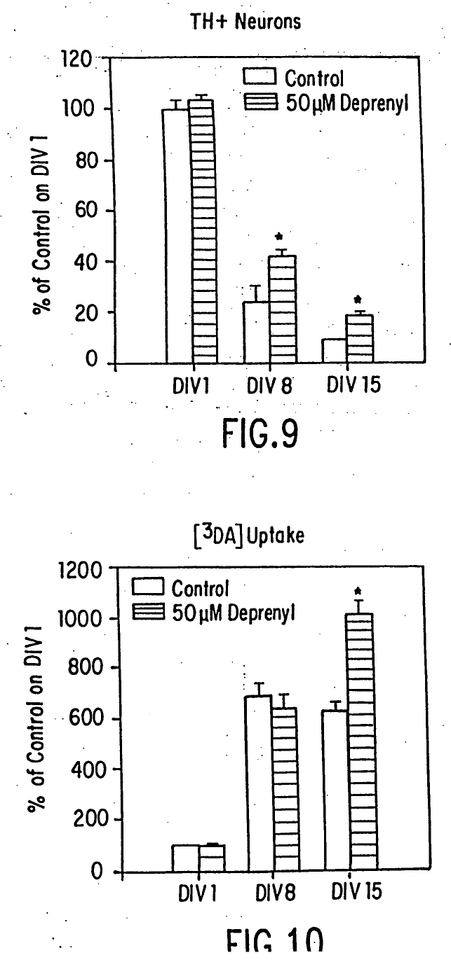

Mesencephalic cultures prepared from embryonic day 14 rats were treated with 0.5, 5 or 50 μM selegiline for 15 days, beginning on the day of plating. (For a more detailed discussion of the culturing of cells and other methods used in these experiments see Mytilineou et al., J. Neurochem.61:1470-1478 (1993)). Survival and growth of dopamine neurons was evaluated by tyrosine hydroxylase (TH) immunocytochemistry and [³H]dopamine uptake and results are shown in FIGS. 9 and 10.

B. Effect of Selegiline on Glutamate Receptor Dependent Cell Death.

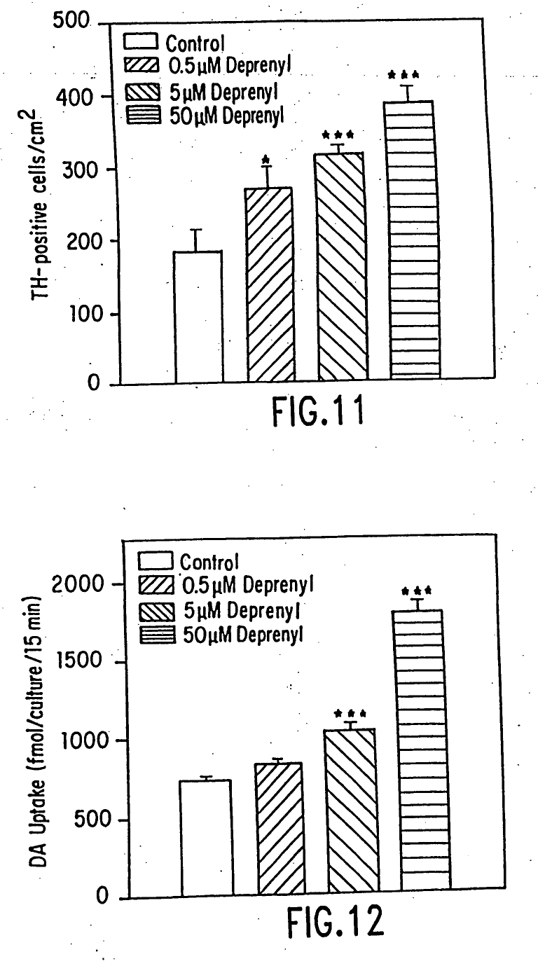

The neuroprotective effect of selegiline was also examined using an experimental paradigm that causes neuronal cell death that can be blocked by inhibition of glutamate receptors. In these experiments, cells were plated and allowed to stabilize for several days. The growth medium of the cells was then changed on a daily basis to induce cell death that can be prevented by blocking glutamate receptors, e.g. using MK-801. After 4 days of daily medium changes, cultures were stained for tyrosine hydroxylase and assayed for uptake of tritiated dopamine. The results shown in FIGS. 11 and 12 further support the conclusion that selegiline promotes the survival of dopaminergic neurons.

C. Effect of Desmethylselegiline on the Survival of Dopamine Neurons.

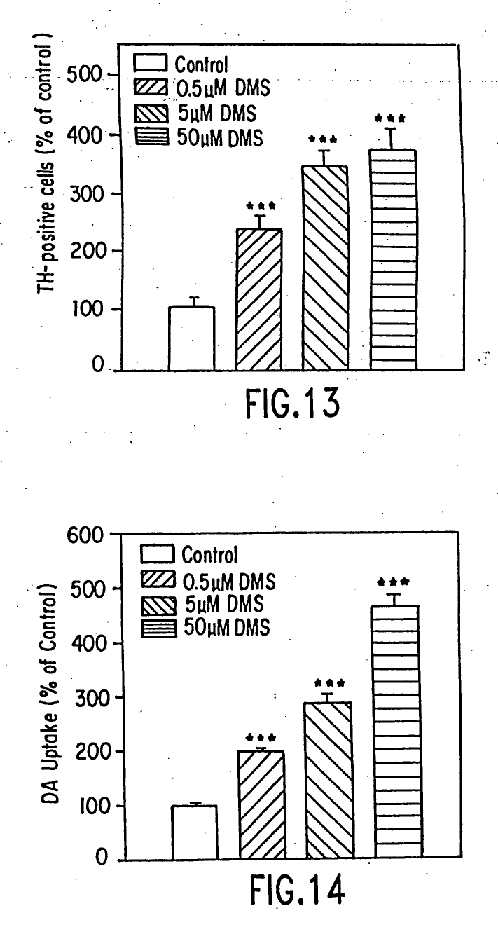

Using the glutamate receptor dependent model of neuron death, an even more potent protection of dopaminergic neurons was provided when desmethylselegiline was used in place of selegiline. Even at the lowest dose tested (0.5 μM), desmethylselegiline caused a significant reduction in the loss of TH positive neurons (FIG. 13) and a significant increase in dopamine uptake (FIG. 14) relative to control cultures in which medium was used without supplementation with either selegiline or desmethylselegiline.

D. Comparison With Other MAO Inhibitors.

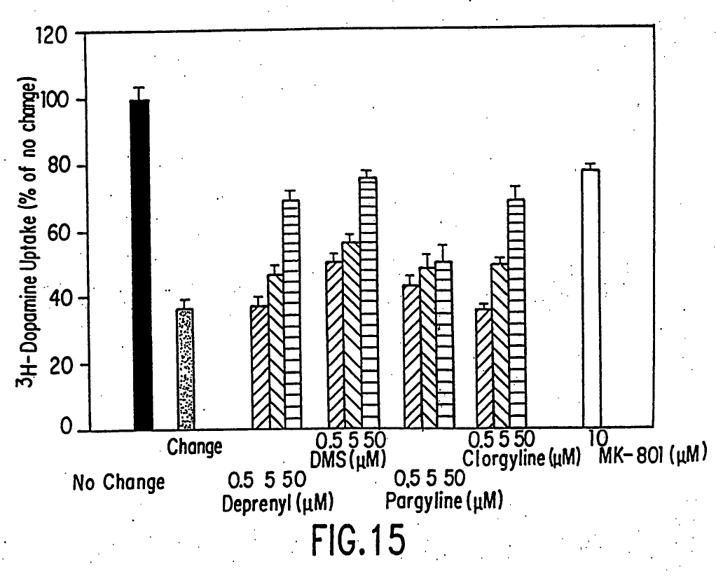

Using the glutamate receptor dependent model of neurotoxicity, the effects of selegiline and desmethylselegiline were compared with two other MAO inhibitors, pargyline and clorgyline (FIG. 15). In agreement with previous results, measurement of dopamine uptake indicated neuron protection by 50 μM deprenyl and 5 and 50 μM desmethylselegiline. Pargyline did not appear to offer any protection at the concentrations used, while clorgyline protected at 50 μM. As expected, protection was also obtained by the NMDA receptor blocker MK-801 (10 μM).

E. Effect of DMS Enantiomers on ³H-Dopamine Uptake.

The data summarized in Table 4 suggests that both (R−)DMS and S(+)DMS are effective as neuroprotectants in mesencephalic dopamine-containing neurons in culture.

TABLE 4
Effect of DMS Enantiomers on Dopamine Uptake

| | ³H-Dopamine uptake |
|---|---|
| Treatment | as a percentage + SEM |
| Control | 100 ± 14.14% |
| R(−)DMS (10 μM) | 140.82 ± 26.20% |
| S(+)DMS (10 μM) | 234 ± 38.36% |

These results were obtained using the medium change model of cell death. Compared to untreated control cells, there was 40% and 134% more axonal growth and terminal axonal survival after treatment with R(−)DMS and S(+)DMS, respectively. In this study, S(+)DMS showed greater potency as a neuroprotectant than R(−)DMS.

### EXAMPLE 7

### Comparison of the Neuroprotective Effect of R(−)DMS and S(+)DMS

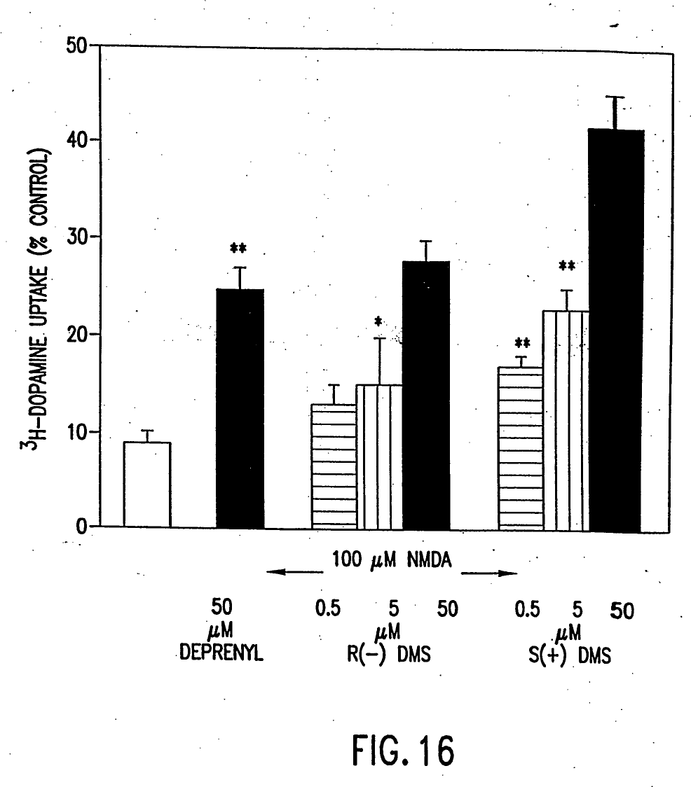

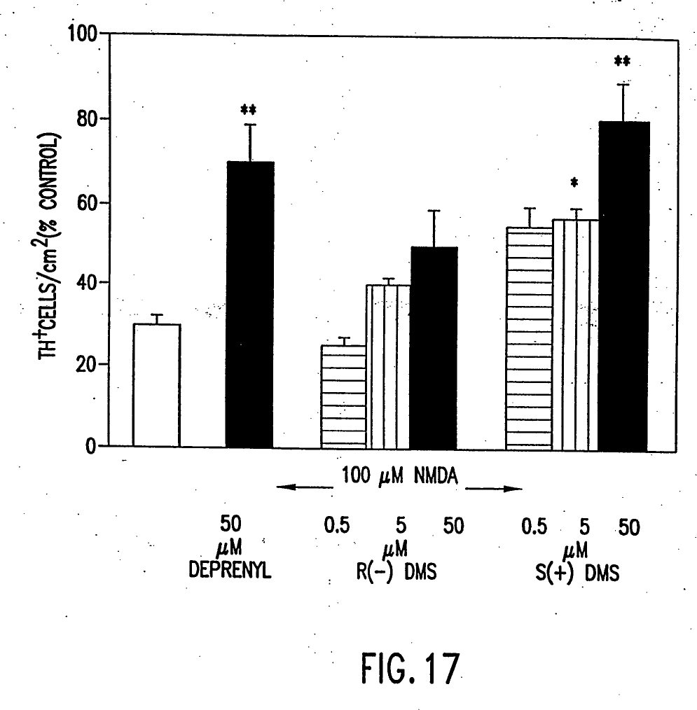

The neuroprotective effect of R(−)DMS and S(+)DMS on cultured rat mesencephalic cells was examined using two models of neuronal cell death. In the first model, cells were exposed to 100 μM N-methyl-D-aspartate (NMDA), an agent which causes cell death by binding to glutamate receptors. Cells exposed to NMDA were incubated in the presence of either medium alone; medium supplemented with 50 μM deprenyl; medium with 0.5, 5, or 50 μM R(−)DMS; or medium containing 0.5, 5 or 50 μM S(+)DMS. The effect of these treatments on ³H-dopamine uptake and the number of TH positive cells was determined and results are shown in Tables 5-8 and FIGS. 16 and 17. It can be seen that both forms of DMS had a neuroprotective effect, with S(+)DMS being the most effective treatment to a statistically significant degree as determined by tritiated dopamine uptake. Experiments examining the neuroprotective effect of DMS enantiomers were also performed using the medium change model of cell death described previously (see Example 6). As can be seen in Tables 9-12, both the R(−) and S(+) enantiomers significantly enhanced [³H)-dopamine uptake and the survival of TH positive cells. In this model, the relative potency of both enantiomers appears to be equal to treatment with 50 μM selegiline.

TABLE 5
R(−)DMS: Dopamine Uptake After 100 μM NMDA Exposure

| | Control | R(−)DMS (0.5 μM) | | R(−)DMS (5.0 μM) | | R(−)DMS (50 μM) | | Deprenyl (50 μM) | |
|---|---|---|---|---|---|---|---|---|---|
| Sample | counts/min | counts/min | % control | counts/min | % control | counts/min | % control | counts/min | % control |
| 1 | 6013 | 9385 | 138.9 | 13509 | 199.9 | 23090 | 341.8 | 18479 | 273.5 |
| 2 | 6558 | 8976 | 132.9 | 11471 | 169.8 | 21530 | 318.7 | 16958 | 251.0 |
| 3 | 7462 | 9028 | 133.6 | 13786 | 204.0 | 17520 | 259.3 | 17550 | 259.8 |
| 4 | 6432 | 8133 | 120.4 | 10229 | 151.4 | 22963 | 339.9 | 18572 | 274.9 |
| 5 | 7317 | 11304 | 167.3 | 11014 | 163.0 | 17708 | 262.1 | 15410 | 228.1 |
| Mean | 6756.4 | 9365.2 | 138.6 | 12001.8 | 177.6 | 20562.2 | 304.3 | 17393.8 | 257.4 |
| St. Dev. | 614.3 | 1177.2 | 17.4 | 1569.7 | 23.2 | 2761.0 | 40.9 | 1295.7 | 19.2 |

TABLE 6
S(+)DMS: Dopamine Uptake After 100 μM NMDA Exposure

| | Control | S(+)DMS (0.5 μM) | | S(+)DMS (5.0 μM) | | S(+)DMS (50 μM) | | Deprenyl (50 μM) | |
|---|---|---|---|---|---|---|---|---|---|
| Sample | counts/min | counts/min | % control | counts/min | % control | counts/min | % control | counts/min | % control |
| 1 | 6013 | 12092 | 179.0 | 20313 | 300.6 | 25944 | 384.0 | 18479 | 273.5 |
| 2 | 6558 | 12269 | 181.6 | 16579 | 245.4 | 28545 | 422.5 | 16958 | 251.0 |
| 3 | 7462 | 16399 | 242.7 | 15929 | 235.8 | 39042 | 577.9 | 17550 | 259.8 |
| 4 | 6432 | 11435 | 169.2 | 15052 | 222.8 | 33024 | 488.8 | 18572 | 274.9 |
| 5 | 7317 | 11096 | 164.2 | 15535 | 229.9 | 25101 | 371.5 | 15410 | 228.1 |
| Mean | 6756.4 | 12658.2 | 187.4 | 16681.6 | 246.9 | 30331.2 | 448.9 | 17393.8 | 257.4 |
| St. Dev. | 614.3 | 2144.9 | 31.7 | 2105.6 | 31.2 | 5764.6 | 85.3 | 1295.7 | 19.2 |

TABLE 7
R(−)DMS: TH Immunochemistry After 100 μM NMDA Exposure

| | Control | R(−)DMS (0.5 μM) | | R(−)DMS (5.0 μM) | | R(−)DMS (50 μM) | | Deprenyl (50 μM) | |
|---|---|---|---|---|---|---|---|---|---|
| Sample | cells/cm² | cells/cm² | % control | cells/cm² | % control | cells/cm² | % control | cells/cm² | % control |
| 1 | 95.0 | 95.0 | 100.9 | 142.5 | 151.3 | 237.5 | 252.2 | 230.0 | 244.2 |
| 2 | 90.0 | 75.0 | 79.6 | 122.5 | 130.1 | 170.0 | 180.5 | 287.5 | 305.3 |
| 3 | 97.5 | 105.0 | 111.5 | 130.0 | 138.1 | 102.5 | 108.8 | 187.5 | 199.1 |
| 4 | | | | 117.5 | 124.8 | 115.0 | 122.1 | 177.5 | 188.5 |
| Mean | 94.17 | 91.67 | 97.3 | 128.13 | 136.1 | 156.25 | 165.9 | 220.63 | 234.3 |
| St. Dev. | 3.8 | 15.3 | 16.2 | 10.9 | 11.5 | 61.6 | 65.4 | 50.1 | 53.2 |

TABLE 8
S(+)DMS: TH Immunochemistry After 100 μM NMDA Exposure

| | Control | S(+)DMS (0.5 μM) | | S(+)DMS (5.0 μM) | | S(+)DMS (50 μM) | | Deprenyl (50 μM) | |
|---|---|---|---|---|---|---|---|---|---|
| Sample | cells/cm² | cells/cm² | % control | cells/cm² | % control | cells/cm² | % control | cells/cm² | % control |
| 1 | 95.0 | 127.5 | 135.4 | 192.5 | 204.4 | 297.5 | 315.9 | 230 | 244.2 |
| 2 | 90.0 | 210 | 223.0 | 187.5 | 199.1 | 202.5 | 215.0 | 287.5 | 305.3 |
| 3 | 97.5 | 177.5 | 188.5 | 192.5 | 204.4 | 317.5 | 337.2 | 187.5 | 199.1 |
| 4 | | | | 172.5 | 183.2 | 222.5 | 236.3 | 177.5 | 188.5 |
| Mean | 94.17 | 171.67 | 182.3 | 186.25 | 197.8 | 260 | 276.1 | 220.63 | 234.3 |
| St. Dev. | | 41.6 | 44.1 | 9.5 | 10.1 | 56.1 | 59.5 | 50.1 | 53.2 |

TABLE 9
R(−)DMS: Dopamine Uptake, Medium Change Model

| | Control | R(−)DMS (0.5 μM) | | R(−)DMS (5.0 μM) | | R(−)DMS (50 μM) | | Deprenyl (50 μM) | |
|---|---|---|---|---|---|---|---|---|---|
| Sample | counts/min | counts/min | % control | counts/min | % control | counts/min | % control | counts/min | % control |
| 1 | 17880 | 29885 | 142.3 | 32577 | 155.2 | 37440 | 178.3 | 38053 | 181.2 |
| 2 | 21500 | 32002 | 152.4 | 29831 | 142.1 | 39200 | 186.7 | 34130 | 162.6 |
| 3 | 23471 | 29934 | 142.6 | 36370 | 173.2 | 39126 | 186.3 | 36810 | 175.3 |
| 4 | 21134 | 27382 | 130.4 | 30342 | 144.5 | 40013 | 190.6 | 33863 | 161.3 |
| Mean | 20996.25 | 29800.75 | 141.9 | 32280 | 153.7 | 38944.75 | 185.5 | 35714 | 170.1 |
| St. Dev. | 2317.2 | 1890.4 | 9.0 | 2976.0 | 14.2 | 1080.7 | 5.1 | 2050.0 | 9.8 |

TABLE 10
S(+)DMS: Dopamine Uptake, Medium Change Model

| | Control | S(+)DMS (0.5 μM) | | S(+)DMS (5.0 μM) | | S(+)DMS (50 μM) | | Deprenyl (50 μM) | |
|---|---|---|---|---|---|---|---|---|---|
| Sample | counts/min | counts/min | % control | counts/min | % control | counts/min | % control | counts/min | % control |
| 1 | 17880 | 35830 | 170.6 | 35976 | 171.3 | 26002 | 123.8 | 38053 | 181.2 |
| 2 | 21500 | 32074 | 152.8 | 36476 | 173.7 | 37320 | 177.7 | 34130 | 162.6 |
| 3 | 23471 | 33042 | 157.4 | 38143 | 181.7 | 30725 | 146.3 | 36810 | 175.3 |
| 4 | 21134 | 39516 | 188.2 | 40964 | 195.1 | 38020 | 181.1 | 33863 | 161.3 |
| Mean | 20996.25 | 35115.5 | 167.2 | 37889.75 | 180.5 | 33016.75 | 157.3 | 35714 | 170.1 |
| St. Dev. | 2317.2 | 3337.9 | 15.9 | 2249.2 | 10.7 | 5715.7 | 27.2 | 2050.0 | 9.8 |

TABLE 11
R(−)DMS: TH Immunochemistry, Medium Change Model

| | Control | R(−)DMS (0.5 μM) | | R(−)DMS (5.0 μM) | | R(−)DMS (50 μM) | | Deprenyl (50 μM) | |
|---|---|---|---|---|---|---|---|---|---|
| Sample | cells/cm² | cells/cm² | % control | cells/cm² | % control | cells/cm² | % control | cells/cm² | % control |
| 1 | 270.0 | 340.0 | 129.0 | 322.5 | 122.3 | 310.0 | 117.6 | 385.0 | 146.0 |
| 2 | 237.0 | 310.0 | 117.6 | 342.5 | 129.9 | 442.5 | 167.9 | 327.5 | 124.2 |
| 3 | 280.0 | 330.0 | 125.2 | 362.5 | 137.5 | 380.0 | 144.1 | 320.0 | 121.4 |
| 4 | 267.5 | 362.5 | | 365.0 | 138.5 | 395.0 | 149.8 | | |
| Mean | 263.63 | 335.63 | 123.9 | 348.13 | 132.1 | 381.88 | 144.9 | 344.17 | 130.6 |
| St. Dev. | 18.6 | 21.8 | 5.8 | 19.8 | 7.5 | 54.8 | 20.8 | 35.6 | 13.5 |

TABLE 12
S(+)DMS: TH Immunochemistry, Medium Change Model

| | Control | S(+)DMS (0.5 μM) | | S(+)DMS (5.0 μM) | | S(+)DMS (50 μM) | | Deprenyl (50 μM) | |
|---|---|---|---|---|---|---|---|---|---|
| Sample | cells/cm² | cells/cm² | % control | cells/cm² | % control | cells/cm² | % control | cells/cm² | % control |
| 1 | 270.0 | 402.5 | 152.7 | 342.5 | 129.9 | 307.5 | 116.6 | 385.0 | 146.0 |
| 2 | 237.0 | 330.0 | 125.2 | 357.5 | 135.6 | 250.0 | 94.8 | 327.5 | 124.2 |
| 3 | 280.0 | 402.5 | 152.7 | 325.0 | 123.3 | 312.5 | 118.5 | 320.0 | 121.4 |
| 4 | 267.5 | 477.5 | | 352.5 | 133.7 | 287.5 | 109.1 | | |
| Mean | 263.6 | 403.1 | 143.5 | 344.4 | 130.6 | 289.4 | 109.8 | 344.2 | 130.6 |
| St. Dev. | 18.6 | 60.2 | 15.9 | 14.3 | 5.4 | 28.4 | 10.8 | 35.6 | 13.5 |

### EXAMPLE 8

### Desmethylselegiline and Ent-Desmethylselegiline as Inhibitors of Dopamine Accumulation (Uptake)

The biological actions of the brain neurotransmitter dopamine are terminated at the synapse by a high-affinity, sodium and energy-dependent transport system (neuronal accumulation or uptake, formerly referred to as “re-uptake”) present within the limiting membrane of the presynaptic dopamine-containing nerve terminal. Inhibition of this transport mechanism would extend the actions of dopamine at the synapse and therefore enhance dopamine synaptic transmission.

A. Method of Testing

The R(−) and S(+) enantiomers of desmethylselegiline (DMS) were tested for their ability to inhibit the dopamine uptake system and compared to selegiline. Inhibitory activity in this assay is indicative of agents of value in the treatment of diseases which require enhanced synaptic dopamine activity. Presently this would include Parkinson's disease, Alzheimer's disease and attention deficit hyperactivity disorder (ADHD).

The assay system used was essentially that described by Fang et al., (Neuro-pharmacology 33:763-768 (1994)). An in vitro nerve-terminal preparation (symptosome-preparation) was obtained from fresh rat neostriatal brain tissue. Transport by dopamine nerve-terminals was estimated by measuring the uptake of tritiated dopamine.

B. Results

As seen in the data presented in Table 13, selegiline, R(−)DMS and S(+)DMS each inhibited dopamine uptake by dopamine-containing nerve terminals. Selegiline and R(−) DMS were approximately equipotent. In contrast, S(+)DMS was 4-5 times more potent than either selegiline or R(−)DMS.

TABLE 13
³H-Dopamine Uptake By Rat Neostriatal Brain Tissue

| | | | % Reduction |
|---|---|---|---|
| Agent | Concentration | | 0 ± SEM |
| Dopamine | 1 | μM | 52.0 ± 4.9 |
| | 10 | μM | 80.9 ± 0.4 |
| Selegiline | 100 | Nm | 7.0 ± 3.6 |
| | 1 | μM | 13.9 ± 4.7 |
| | 10 | μM | 16.3 ± 3.8 |
| | 100 | μM | 59.8 ± 1.0 |
| R(−)DMS | 100 | nM | 11.5 ± 1.0 |
| | 1 | μM | 10.7 ± 2.8 |
| | 10 | μM | 20.1 ± 3.1 |
| | 100 | μM | 51.3 ± 2.6 |
| S(+)DMS | 100 | nM | 15.3 ± 7.7 |
| | 1 | μM | 24.1 ± 11.7 |
| | 10 | μM | 47.0 ± 3.1 |
| | 100 | μM | 76.9 ± 1.8 |

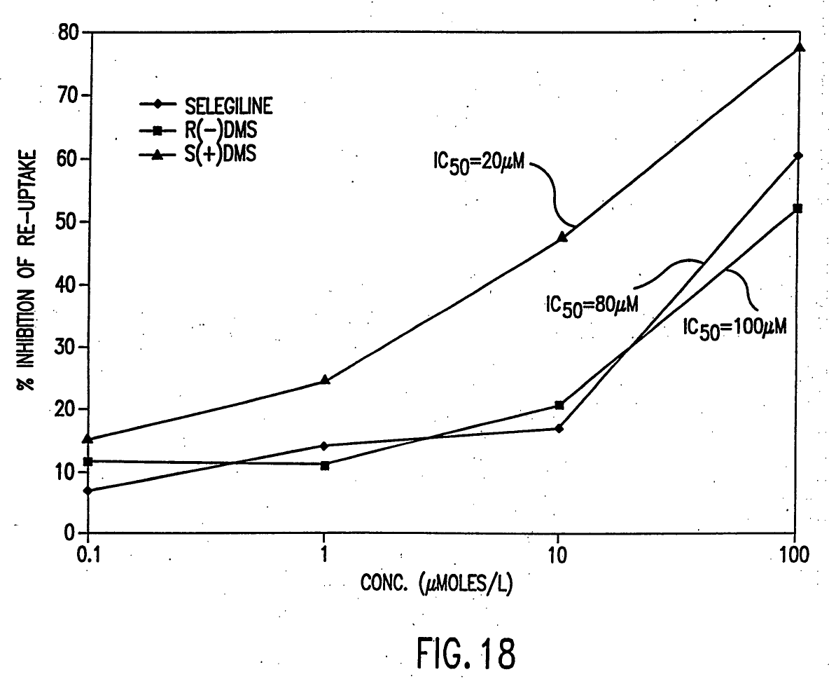

Relative potency can be expressed in terms of the concentration required to inhibit dopamine uptake by 50% (IC₅₀). The ICso values were determined graphically (see FIG. 18) and are shown below in Table 14.

TABLE 14
Concentrations Needed to Inhibit Dopamine Uptake by 50%

| Agent | IC₅₀ | Relative Potency |
|---|---|---|
| Selegiline | ≈80 μM | 1 |
| R(−)DMS | ≈100 μM | 0.8 |
| S(+)DMS | ≈20 μM | 4 |

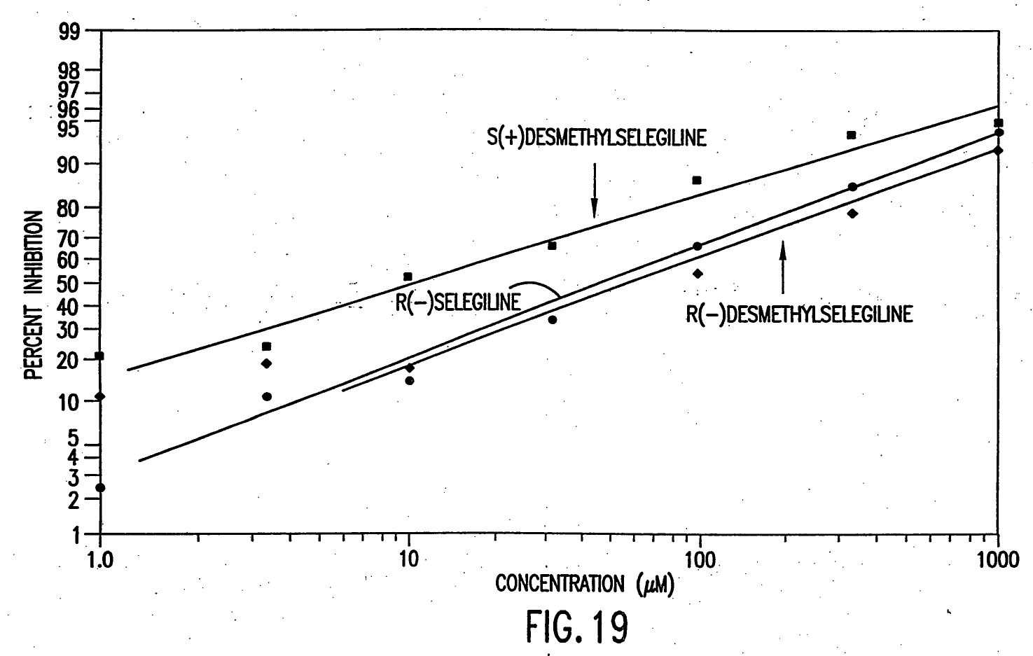

The experiment described above was repeated in a concentration range designed to more accurately describe IC₅₀ values and results are shown in FIG. 19. ID₅₀ values determined based upon the graph are shown in Table 15.

TABLE 15
Concentrations Needed to Inhibit Dopamine Uptake by 50%

| | | Potency Relative to |
|---|---|---|
| Compound | IC₅₀ | Selegiline |
| S(+)DMS | 11 μM | 4.2 |
| selegiline | 46 μM | 1 |
| R(−)DMS | 54 μM | 1.2 |

C. Conclusions

The results demonstrate that, at the appropriate concentration, selegiline and each of the enantiomers of DMS inhibit transport of released dopamine at the neuronal synapse and enhance the relative activity of this neurotransmitter at the synapse. In this regard, S(+)DMS is more potent than selegiline which, in turn, is more potent than R(−)DMS. Of the agents tested, S(+)DMS is the most preferred with regard to the treatment of hypodopaminergic conditions such as ADHD.

### EXAMPLE 9

### Actions of the R(−) and S(+) enantiomers of Desmethylselegiline (DMS) on Human Platelet MAO-B and Guinea Pig Brain MAO-B and MAO-A Activity

Human platelet MAO is comprised exclusively of the type-B isoform of the enzyme. In the present study, the in vitro and in vivo inhibition of this enzyme by the two enantiomers of DMS was determined and compared with inhibition due to selegiline. The present study also examined the two enantiomers of DMS for inhibitory activity with respect to the MAO-A and MAO-B in guinea pig hippocampal tissue. Guinea pig brain tissue is an excellent animal model for studying brain dopamine metabolism, the enzyme kinetics of the multiple forms of MAO and the inhibitory properties of novel agents that interact with these enzymes. The multiple forms of MAO in this animal species show similar kinetic properties to those found in human brain tissue. Finally, the test agents were administered to guinea pigs and the extent to which they might act as inhibitors of brain MAO in vivo was assessed.

A. Method of Testing

In vitro: The test system utilized the in vitro conversion of specific substrates of MAO-A (¹⁴C-serotonin) in guinea pig hippocampal homogenates or MAO-B (¹⁴C-phenylethylamine) by human platelets and guinea pig hippocampal homogenates. The rate of conversion of each substrate was measured in the presence of S(+)DMS, R(−)DMS or selegiline and compared to the isozyme activity in the absence of these agents. A percent inhibition was calculated from these values. Potency was evaluated by comparing the concentration of each agent which caused a 50% inhibition(IC₅₀ value).

In vivo: R(−)DMS, S(+)DMS or selegiline was administered in vivo subcutaneously (sc), once a day for 5 days prior to sacrifice. Hippocampal homogenates containing enzyme were prepared, and assays in vitro for MAO-A and MAO-B activity. These experiments were performed to demonstrate that the DMS enantiomers were capable of entering brain tissue and inhibiting MAO activity.

B. Results

MAO-B Inhibitory Activity In Vitro

Results for MAO-B inhibition are shown in Tables 16 and 17. IC₅₀ values for MAO-B inhibition and potency as compared to selegiline is shown in Table 18.

TABLE 16
MAO-B Inhibition in Human Platelets Concentration

| | | | % Inhibition |
|---|---|---|---|
| Agent | Concentration | | 0 ± SEM |
| Selegiline | 0.3 | nM | 8.3 ± 3.4 |
| | 5 | nM | 50.3 ± 8.7 |
| | 10 | nM | 69.0 ± 5.5 |
| | 30 | nM | 91.0 ± 1.4 |
| | 100 | nM | 96.0 ± 1.6 |
| | 300 | nM | 96.0 ± 1.6 |
| | 1 | μM | 96.6 ± 1.6 |
| R(−)DMS | 100 | nM | 14.3 ± 3.6 |
| | 300 | nM | 42.1 ± 4.0 |
| | 1 | μM | 76.9 ± 1.47 |
| | 3 | μM | 94.4 ± 1.4 |
| | 10 | μM | 95.8 ± 1.4 |
| | 3 | μM | 95.7 ± 2.3 |
| S(+)DMS | 300 | nM | 6.4 ± 2.8 |
| | 1 | μM | 11.1 ± 1.0 |
| | 3 | μM | 26.6 ± 1.9 |
| | 10 | μM | 42.3 ± 2.3 |
| | 30 | μM | 68.2 ± 2.34 |
| | 100 | μm | 83.7 ± 0.77 |
| | 1 | mM | 94.2 ± 1.36 |

TABLE 17
MAO-B Inhibition in Guinea Pig Hippocampus

| | | | % Inhibition |
|---|---|---|---|
| Agent | Concentration | | 0 ± SEM |
| Selegiline | 0.3 | μM | 28.3 ± 8.7 |
| | 5 | nM | 81.2 ± 2.6 |
| | 10 | nM | 95.6 ± 1.3 |
| | 30 | nM | 98.5 ± 0.5 |
| | 100 | nM | 98.8 ± 0.5 |
| | 300 | nM | 98.8 ± 0.5 |
| | 1 | μM | 99.1 ± 0.45 |
| R(−)DMS | 100 | nM | 59.4 ± 9.6 |
| | 300 | nM | 86.2 ± 4.7 |
| | 1 | μM | 98.2 ± 0.7 |
| | 3 | μM | 98.4 ± 0.95 |
| | 10 | μm | 99.1 ± 0.45 |
| | 30 | μM | 99.3 ± 0.40 |
| S(+)DMS | 300 | nM | 18.7 ± 2.1 |
| | 1 | μM | 44.4 ± 6.4 |
| | 3 | μM | 77.1 ± 6.0 |
| | 10 | μM | 94.2 ± 1.9 |
| | 30 | μM | 98.3 ± 0.6 |
| | 100 | μM | 99.3 ± 0.2 |
| | 1 | μm | 99.9 ± 0.1 |

TABLE 18
IC₅₀ Values for the Inhibition of MAO-B Guinea Pig

| | | Guinea Pig |
|---|---|---|
| Treatment | Human Platelets | Hippocampal Cortex |
| Selegiline | 5 nM (1) | 1 nM (1) |
| R(−)DMS | 400 nM (80) | 60 nM (60) |
| S(+)DMS | 1400 nM (2800) | 1200 nM (1200) |

( ) = reduction in potency compared to selegiline

As observed, R(−)DMS was 20-35 times more potent than S(+)DMS as an MAO-B inhibitor and both enantiomers were less potent than selegiline.

MAO-A Inhibitory Activity In Vitro

Results obtained from experiments examining the inhibition of MAO-A in guinea pig hippocampus are summarized in Table 19. The IC₅₀ values for the two enantiomers of DMS and for selegiline are shown in Table 20.

TABLE 19
MAO-A Inhibition in Guinea Pig Hippocampus

| | | | % Reduction |
|---|---|---|---|
| Agent | Concentration | | 0 ± SEM |
| Selegiline | 300 | nM | 11.95 ± 2.4 |
| | 1 | μM | 22.1 ± 1.2 |
| | 3 | μM | 53.5 ± 2.7 |
| | 10 | μM | 91.2 ± 1.16 |
| | 100 | μM | 98.1 ± 1.4 |
| | 1 | mM | 99.8 ± 0.2 |
| R(−)DMS | 300 | nM | 4.8 ± 2.1 |
| | 1 | μM | 4.2 ± 1.5 |
| | 3 | μM | 10.5 ± 2.0 |
| | 10 | μM | 19.0 ± 1.3 |
| | 100 | μM | 64.2 ± 1.5 |
| | 1 | mM | 96.5 + 1.2 |
| S(+)DMS | 1 | μM | 3.3 + 1.5 |
| | 3 | μM | 4.3 ± 1.0 |
| | 10 | μM | 10.5 ± 1.47 |
| | 100 | μM | 48.4 ± 1.8 |
| | 1 | nM | 92.7 ± 2.5 |
| | 10 | nM | 99.6 ± 0.35 |

TABLE 20
IC₅₀ Values for the Inhibition of MAO-A

| | IC₅₀ for MAO-A in Guinea |
|---|---|
| Treatment | Pig Hippocampal Cortex |
| Selegiline | 2.5 μM (1) |
| R(−)DMS | 50.0 μM (20) |
| S(+)DMS | 100.0 μM (40) |

( ) = reduction in potency compared to selegiline

R(−)DMS was twice as potent as S(+)DMS as an MAO-A inhibitor and both were 20-40 times less potent than selegiline. Moreover, each of these agents were 2-3 orders of magnitude, i.e., 100 to 1000 times, less potent as inhibitors of MAO-A than inhibitors of MAO-B in hippocampal brain tissue. Therefore, selegiline and each enantiomer of DMS can be classified as selective MAO-B inhibitors in brain tissue.

Results of In Vivo Experiments

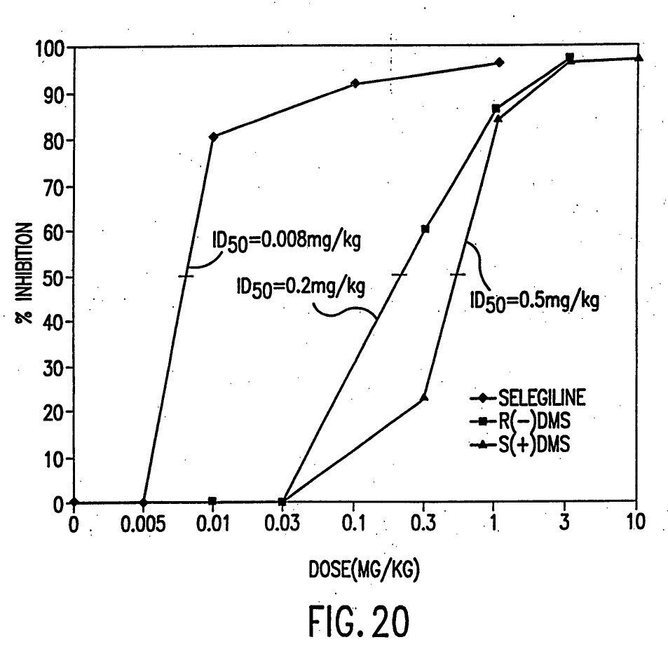

Each enantiomer of DMS was administered in vivo by subcutaneous injection once a day for five consecutive days, and inhibition of brain MAO-B activity was then determined. In preliminary studies, selegiline was found to have an ID50 of 0.03 mg/kg; and both R(−)DMS and S(+)DMS were determined to be about 10 times less potent. More recent studies, performed on a larger group of animals, indicates that R(−)DMS is actually about 25 times less potent than selegiline as an inhibitor of MAO-B and that S(+)DMS is about 50 times less potent. Results are shown in FIG. 20 and ID50 values are summarized in Table 21.

TABLE 21
ID₅₀ Values for Brain MAO-B Following 5 Days of Administration

| | ID₅₀ for MAO-B in Guinea |
|---|---|
| Treatment | Pig Hippocampal Cortex |
| Selegiline | 0.008 mg/kg (1) |
| R(−)DMS | 0.20 mg/kg (25) |
| S(+)DMS | 0.50 mg/kg (60) |

( ) - reduction in potency compared to selegiline

This experiment demonstrates that the enantiomers of DMS penetrate the blood brain-barrier and inhibit brain MAO-B after in vivo administration. It also demonstrates that the potency differences as an MAO-B inhibitor observed in vitro between each of the DMS enantiomers and selegiline are substantially reduced under in vivo conditions.

In experiments examining the effect of 5 s.c. treatments on MAO-A activity in guinea pig cortex (hippocampus), it was found that selegiline administration at a dose of 1.0 mg/kg resulted in a 36.1% inhibition of activity. R(−)DMS resulted in an inhibition of 29.8% when administered at a dose of 3.0 mg/kg. S(+)DMS administration did not cause any observable inhibition at the highest dose tested (10 mg/kg) indicating that it has significantly less cross reactivity potential.

C. Conclusions

In vitro, R(−)DMS and S(+)DMS both exhibit activity as MAO-B and MAO-A inhibitors. Each enantiomer was selective for MAO-B. S(+)DMS was less potent than R(−)DMS and both enantiomers of DMS were less potent than selegiline in inhibiting both MAO-A and MAO-B.

In vivo, both enantiomers demonstrated activity in inhibiting MAO-B, indicating that these enantiomers are able to cross the blood-brain barrier. The ability of these agents to inhibit MAO-B suggests that these agents may be of value as therapeutics for hypodopaminergic diseases such as ADHD and dementia.

### EXAMPLE 10

### Examples of Dosage Forms

A. Desmethylselegiline Patch.

| Dry Weight Basis | |
|---|---|
| Component | (mg/cm²) |
| Durotak ® 87-2194 | 90 parts by weight |
| adhesive acrylic polymer | |
| Desmethylselegiline | 10 parts by weight |

The two ingredients are thoroughly mixed, cast on a film backing sheet (e.g., Scotchpak® 9723 polyester) and dried. The backing sheet is cut into patches a fluoropolymer release liner (e.g., Scotchpak® 1022) is applied, and the patch is hermetically sealed in a foil pouch. One patch is applied daily to supply 1-10 mg of desmethylselegiline per 24 hours in the treatment of conditions in a human produced by neuronal degeneration or neuronal trauma.

B. Intravenous Solution.

A 1% solution is prepared by dissolving 1 g of desmethylselegiline as the HCl in sufficient 0.9% isotonic saline solution to provide a final volume of 100 ml. The solution is buffered to pH 4 with citric acid, sealed, and sterilized to provide a 1% solution suitable for intravenous administration in the treatment of conditions produced by neuronal degeneration, neuronal trauma, or neuronal damage caused by stroke.

C. Oral Dosage Form

Tablets and capsules containing desmethylselegiline are prepared from the following ingredients (mg/unit dose):

| | |
|---|---|
| desmethylselegiline | 1-5 |
| microcrystalline cellulose | 86 |
| lactose | 41.6 |
| citric acid | 0.5-2 |
| sodium citrate | 0.1-2 |
| magnesium stearate | 0.4 |

with an approximately 1:1 ratio of citric acid and sodium citrate.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method for the treatment of stroke in a subject in need of such treatment comprising:
administering R(−)-desmethylselegiline to the subject in an amount sufficient to limit, reduce or eliminate neuronal damage associated with the stroke.

2. The method of claim 1, wherein the stroke is ischemic stroke.

3. The method of claim 1, wherein the stroke is a transient ischemic attack.

4. The method of claim 1, wherein the stroke is an intracranial hemorrhage.

5. The method of claim 4, wherein the intracranial hemorrhage is a parenchymatous hemorrhage or a subarachnoid hemorrhage.

6. The method of claim 4, wherein the intracranial hemorrhage is caused by an aneurysm.

7. The method of claim 6, wherein the aneurysm is a saccular aneurysm.

8. The method of claim 1, wherein the stroke is caused by a drug, fibromuscular dysplasia, arterial dissection, homocystinuria, migraine, or emboli.

9. The method of claim 1, further comprising administering a therapeutic agent useful in the treatment of stroke in conjunction with R(−)-desmethylselegiline.

10. The method of claim 9, wherein the therapeutic agent is selected from the group consisting of tissue plasminogen activator, aspirin, and heparin.

11. The method of claim 1, wherein the subject is a human.

12. The method of claim 1, wherein the R(−)-desmethylselegiline is administered orally.

13. The method of claim 1, wherein the R(−)-desmethylselegiline is administered by a route that avoids absorption of R(−)-desmethylselegiline from the gastrointestinal tract.

14. The method of claim 13, wherein the R(−)-desmethylselegiline is administered intravenously, transdermally, buccally, sublingually, or parenterally.

15. The method of claim 1, wherein the R(−)-desmethylselegiline is administered at a dose of between 0.01 mg/kg per day and 0.15 mg/kg per day.

16. A method for the treatment of cerebral edema in a subject in need of such treatment, comprising:
administering R(−)-desmethylselegiline to the subject in an amount sufficient to limit, reduce or eliminate neuronal damage associated with the cerebral edema.

17. The method of claim 16, wherein the cerebral edema is intracellular edema.

18. The method of claim 16, wherein the cerebral edema is interstitial edema.

19. The method of claim 16, wherein the subject is a human.

20. The method of claim 16, wherein the R(−)-desmethylselegiline is administered orally.

21. The method of claim 16, wherein the R(−)-desmethylselegiline is administered by a route that avoids absorption of R(−)-desmethylselegiline from the gastrointestinal tract.

22. The method of claim 21, wherein the R(−)-desmethylselegiline is administered intravenously, transdermally, buccally, sublingually, or parenterally.

23. The method of claim 16, wherein the R(−)-desmethylselegiline is administered at a dose of between 0.01 mg/kg per day and 0.15 mg/kg per day.

24. A method for the treatment of cerebrovascular disease in a subject in need of such treatment, comprising:
administering R(−)-desmethylselegiline to the subject in an amount sufficient to limit, reduce or eliminate neuronal damage associated with the cerebrovascular disease.

25. The method of claim 24, wherein the cerebrovascular disease is selected from the group consisting of stroke, intracranial hemorrhage, occlusive hemorrhage, cerebral hemorrhage, subarachnoid hemorrhage, hemorrhagic lesion, subderal hematoma, aneurysm, and cerebral abscess.

26. The method of claim 24, wherein the cerebrovascular disease is caused by cerebral ischemia.

27. The method of claim 26, wherein the cerebral ischemia causes selective ischemic necrosis or cerebral infarction.

28. The method of claim 24, wherein the cerebrovascular disease is caused by cerebral hypoxia.

29. The method of claim 24, wherein the cerebrovascular disease is caused by traumatic brain injury, atherosclerosis, vasculitide, or arrhythmia.

30. The method of claim 29, wherein the arrhythmia is atrial fibrillation.

31. The method of claim 24, wherein the subject is a human.

32. The method of claim 24, wherein the R(−)-desmethylselegiline is administered orally.

33. The method of claim 24, further comprising administering a therapeutic agent useful in the treatment of cerebrovascular disease in conjunction with R(−)-desmethylselegiline to the subject.

34. The method of claim 33, wherein the therapeutic agent is selected from the group consisting of tissue plasminogen activator, aspirin, heparin, heparinoids, ticlopidine, clopidogrel, warfarin, glutamate receptor antagonists, nimodipine, phenylephrine, and dopamine.

35. The method of claim 24, wherein the R(−)-desmethylselegiline is administered by a route that avoids absorption of R(−)-desmethylselegiline from the gastrointestinal tract.

36. The method of claim 35, wherein the R(−)-desmethylselegiline is administered intravenously, transdermally, buccally, sublingually, or parenterally.

37. The method of claim 24, wherein the R(−)-desmethylselegiline is administered at a dose of between about 0.01 mg/kg per day and about 0.15 mg/kg per day.
